(19) United States
(12) Patent Application Publication
(10) Pub. No.: US 2023/0310053 A1
(43) Pub. Date: Oct. 5, 2023

(54) SYSTEMS AND METHODS FOR PERFORMING A DENERVATION PROCEDURE AND DETERMINING THE EFFICACY THEREOF

(71) Applicant: Otsuka Medical Devices Co., Ltd., Tokyo (JP)

(72) Inventors: Andrew Wu, Los Altos Hills, CA (US); Neil Barman, Menlo Park, CA (US)

(21) Appl. No.: 18/183,792

(22) Filed: Mar. 14, 2023

Related U.S. Application Data
(60) Provisional application No. 63/319,912, filed on Mar. 15, 2022.

(51) Int. Cl. A61B 18/00

(52) U.S. Cl. CPC A61B 18/00; A61B 2018/00404; A61B 2018/00434; A61B 2018/00511; A61B 2018/00577; A61B 2018/00642; A61B 2018/00839

(57) ABSTRACT

Described herein are systems and methods for performing a denervation procedure and determining an efficacy thereof. Such a system can include an excitation source, a controller, and a catheter with element(s) for delivering first ablation therapy from a first longitudinal location along a biological lumen and delivering second ablation therapy from a second longitudinal location longitudinally spaced apart from the first longitudinal location. A sensing subsystem of the system senses neural activity from a third longitudinal location along the biological lumen, to determine the efficacy of at least one of the first or second ablation therapies.

### PRIORITY CLAIM

This application claims priority to U.S. Provisional Pat. Application No. 63/319,912, filed Mar. 15, 2022, titled METHODS AND SYSTEMS FOR MEASURING NEURAL ACTIVITY BETWEEN PAIR OF ABLATION SITES TO DETERMINE EFFICACY OF DENERVATION PROCEDURE, which is incorporated herein by reference.

### TECHNICAL FIELD

Embodiments of the present technology generally relate to techniques for performing a denervation procedure and determining the efficacy of the denervation procedure, and related systems and methods.

### BACKGROUND

The human body’s nervous system includes both the somatic nervous system that provides sense of the environment (vision, skin sensation, etc.) and regulation of the skeletal muscles, and is largely under voluntary control, and the autonomic nervous system, which serves mainly to regulate the activity of the internal organs and adapt them to the body’s current needs, and which is largely not under voluntary control. The autonomic nervous system involves both afferent or sensory nerve fibers that can mechanically and chemically sense the state of an organ, and efferent fibers that convey the central nervous system’s response (sometimes called a reflex arc) to the sensed state information. In some cases, the somatic nervous system is also influenced, such as to cause vomiting or coughing in response to a sensed condition.

Regulation of the human body’s organs can therefore be somewhat characterized and controlled by monitoring and affecting the nerve reflex arc that causes organ activity. For example, the renal nerves leading to a kidney can often cause a greater reflexive reaction than desired, contributing significantly to hypertension. Measurement of the nerve activity near the kidney, and subsequent ablation of renal nerves can therefore be used to control the nervous system’s overstimulation of the kidney, improving operation of the kidney and the body as a whole.

Because proper operation of the nervous system is therefore an important part of proper organ function, it is desired to be able to monitor and change nervous system function in the human body to characterize and correct nervous system regulation of internal human organs.

New medical therapies have been practiced whereby a catheter is inserted into the body to a specified anatomical location and destructive means are conveyed to nerves by means of the catheter (aka probe) to irreversibly damage tissue in the nearby regions. The objective is to modulate (e.g., abolish) nerve function in the specified anatomic location. The result is that abnormally functioning physiological processes can be terminated or modulated back into a normal range. Unfortunately, such medical therapies are not always successful because of an inability to assess that the neural activity has been successfully abolished. An alternative objective can be to increase a physiologic process or modulate it to an abnormal range.

An example is renal nerve ablation, which is also known to as renal denervation, to relieve hypertension. Various studies have confirmed the relationship of renal nerve activity with blood pressure regulation. In various renal ablation procedures, a catheter is introduced into a hypertensive patient’s arterial vascular system and advanced into the renal artery. Renal nerves are located in the arterial wall and/or in regions adjacent to the artery. Destructive means are delivered proximate to the renal artery wall to an extent intended to cause destruction of renal nerve activity. Destructive means include energy such as radiofrequency (RF), microwave, cryotherapy, ultrasound, optical, laser or chemical agents. The objective is to abolish the renal nerve activity. Such nerve activity is an important factor in the creation and/or maintenance of hypertension and abolishment of the nerve activity reduces blood pressure and/or medication burden.

Unfortunately not all patients respond to this therapy. Renal nerve ablation procedures are often ineffective, potentially due to a poor probe/tissue interface. Accordingly, insufficient quantities of destructive means are delivered to the nerve fibers transmitting along the renal artery. One reason is that the delivery of destructive means to the arterial wall does not have a feedback mechanism to assess efficacy of the destruction of the nerve activity. As a consequence an insufficient quantity of destructive means is delivered and nervous activity is not abolished. Clinicians, therefore, would benefit from improved ways to monitor the integrity of the nerve fibers passing through the arterial wall in order to confirm destruction of nerve activity prior to terminating therapy. Current technology for the destruction of nerve activity does not provide practitioners with a feedback mechanism to detect when the desired nervous activity destruction is accomplished. Nerve destructive means are applied empirically without knowledge that the desired effect has been achieved.

It is known that ablation of the renal nerves, with sufficient energy, is able to effect a reduction in both systolic and diastolic blood pressure. Current methods are said to be, from an engineering perspective, open loop; i.e., the methods used to effect renal denervation do not employ any way of measuring, in an acute clinical setting, the results of applied ablation energies. It is only after application of such energies and a period of time (3-12 months) that the effects of the procedure are known.

### SUMMARY

Certain embodiments of the present technology relates to system for performing a denervation procedure and determining an efficacy thereof. In accordance with certain embodiments, such a system comprises a catheter including an element or first and second elements configured to selectively deliver ablation therapy and one or more sensing electrodes configured to selectively sense neural activity. The system also comprises an excitation source configured to selectively energize the element or the first and second elements of the catheter to thereby cause the element or the first and second elements to deliver the ablation therapy. Additionally, the system comprises a sensing subsystem electrically coupled to the at least one sensing electrode. The system further comprises a controller communicatively coupled to the catheter, the excitation source and the sensing subsystem. In accordance with certain embodiments, the controller is configured to cause the excitation source to energize the element or the first element to thereby delivery first ablation therapy, while the element or the first element is located at a first longitudinal location along a biological lumen. The controller is also configured to cause the excitation source to energize the element or the second element to thereby delivery second ablation therapy, while the element or the second element is located at a second longitudinal location along the biological lumen that is longitudinally spaced apart from the first longitudinal location. The controller is further configured to cause the sensing subsystem to sense neural activity from a third longitudinal location, using at least one of the one or more sensing electrodes to determine the efficacy of at least one of the first or second ablation therapies.

Certain embodiments of the present technology are directed to methods for performing a denervation procedure and determining an efficacy thereof. Such a method can include delivering first ablation therapy from a first longitudinal location along a biological lumen, and delivering second ablation therapy from a second longitudinal location longitudinally spaced apart from the first longitudinal location along the biological lumen. The method further includes sensing neural activity from a third longitudinal location that is longitudinally located between the first and the second longitudinal locations, from which the first and the second ablation therapy were respectively delivered to determine the efficacy of at least one of the first or second ablation therapies.

In accordance with certain embodiments, a method includes delivering first ablation therapy from a first longitudinal location along a biological lumen, delivering second ablation therapy from a second longitudinal location longitudinally spaced apart from the first longitudinal location along the biological lumen, and delivering stimulation energy using an electrode that is located at a third longitudinal location along the biological lumen, wherein the third longitudinal location is one of proximal or distal to both the first and the second longitudinal locations. The method also includes sensing an evoked neural response to the stimulation energy using a further electrode located at a fourth longitudinal location along the biological lumen, wherein the fourth longitudinal location is the other one of proximal or distal to both the first and the second longitudinal locations. The method further includes determining, based on the evoked neural response that is sensed, whether or not further ablation therapy should be delivered from a location along a longitudinal length of the biological lumen.

This summary is not intended to be a complete description of the embodiments of the present technology. Other features and advantages of the embodiments of the present technology will appear from the following description in which the preferred embodiments have been set forth in detail, in conjunction with the accompanying drawings and claims.

### BRIEF DESCRIPTION OF THE FIGURES

FIG. 1 illustrates a kidney that receives blood via the abdominal aorta, a renal artery, and branch vessels.

FIG. 2 is a cross-section of a renal artery and is used to describe the various layers and nerves of the renal artery wall.

FIG. 3 illustrates how a transducer within a renal artery can be used to deliver ablation therapy to a treatment region as part of a denervation procedure, and is used to describe why it is difficult to determine the efficacy of such a denervation procedure.

FIG. 4 illustrates an improved technique for determining the efficacy of a denervation procedure that involves performing nerve destruction at two different longitudinal locations along a renal artery and then sensing for a native or evoked neural response at a longitudinal location between the two nerve destruction sites.

FIG. 5 illustrates expected sensed nerve activity at three regions (labeled A, B, and C in FIG. 4) following the denervation procedure described with reference to FIG. 4.

FIG. 6 is a high level flow diagram that is used to summarize methods for performing a denervation procedure and determining an efficacy thereof, according to certain embodiments of the present technology.

FIG. 7A shows an example catheter with its two selectively deployable electrodes in their non-deployed positions.

FIG. 7B shows the catheter, which was introduced in FIG. 7A, with its two selectively deployable electrodes in their deployed positions.

FIG. 8 is a schematic diagram of an example system, according to an embodiment of the present technology, for interfacing with a patient’s arterial nerves.

FIGS. 9A and 9B illustrate example cross-sections of a portion of the shaft of the catheter shown in FIGS. 7A and 7B.

FIG. 10 illustrates example details of a fluid supply subsystem introduced in FIG. 8.

FIGS. 11A and 11B illustrates, respectively, a longitudinal cross-sectional view and a radial cross-sectional view of an example transducer of the catheter shown in FIGS. 7A and 7B.

### DETAILED DESCRIPTION

In the following detailed description of example embodiments, reference is made to specific example embodiments by way of drawings and illustrations. These examples are described in sufficient detail to enable those skilled in the art to practice what is described, and serve to illustrate how elements of these examples may be applied to various purposes or embodiments. Other embodiments exist, and logical, mechanical, electrical, and other changes may be made. Features or limitations of various embodiments described herein, however important to the example embodiments in which they are incorporated, do not limit other embodiments, and any reference to the elements, operation, and application of the examples serve only to define these example embodiments. Features or elements shown in various examples described herein can be combined in ways other than shown in the examples, and any such combination is explicitly contemplated to be within the scope of the examples presented here. The following detailed description does not, therefore, limit the scope of what is claimed.

Regulating operation of the nervous system to characterize nerve signaling and modulate organ function includes in some examples introduction of a catheter (aka probe) into the body to a specified anatomical location, and partially destroying or ablating nerves using the probe to destroy nerve tissue in the region near the probe. By reducing nerve function in the selected location, an abnormally functioning physiological process can often be regulated back into a normal range. It would also be possible to modulate nerve function to purposely cause an abnormally functioning physiological process that is beneficial to the patient.

Unfortunately, it is typically very difficult to estimate the degree to which nerve activity has been reduced, which makes it difficult to perform a procedure where it is desired to ablate all nerves, or to ablate some, but not all, nerves to bring the nervous system response back into a desired range without destroying the nervous system response entirely. A denervation procedure may be used, for example, to perform renal nerve ablation to treat hypertension, as was noted above.

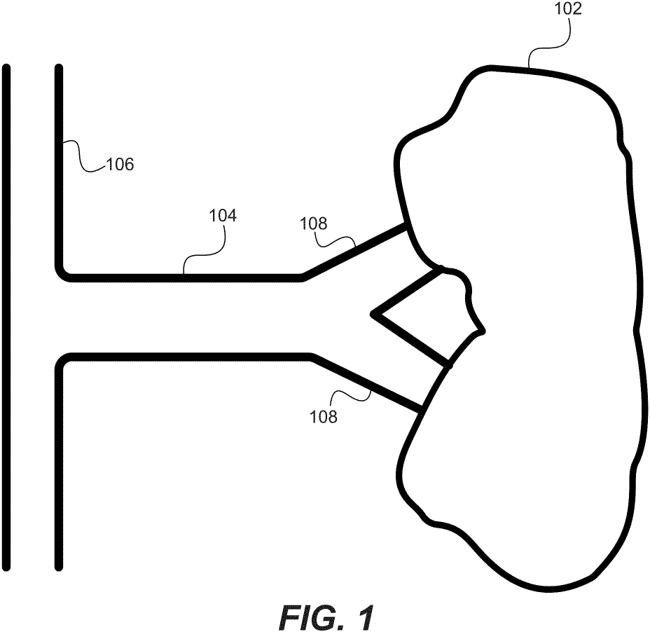

As illustrated in FIG. 1, a kidney 102 receives blood via the abdominal aorta 106, a renal artery 104, and branch vessels 108. Renal sympathetic nerves, which can also be referred to as sympathetic renal nerves, generally follow the abdominal aorta 106 and the renal artery 104 allowing for communication between the brain and the kidney 102. The sympathetic renal nerves include both the afferent sensory renal nerves that carry neural impulses from the kidney 102 to the brain, and the efferent sympathetic renal nerves that carry neural impulses from the brain to the kidney 102. In other words, efferent renal nerves that follow the renal artery 104 carry impulses from the brain to the kidney 102. By contrast, afferent renal nerves that follow the renal artery carry impulses from the kidney 102 to the brain.

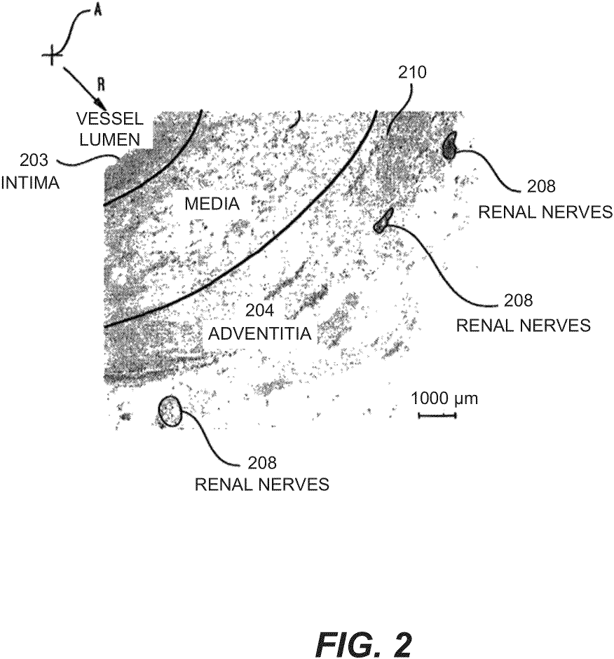

As illustrated in FIG. 2, which is a cross-section of the renal artery 104, the renal artery wall is made up of multiple layers, including: the intima 203, which includes an inner single layer of endothelial cells; the media 205, which is in the center of the artery wall; and the adventitia 204, which is the outside layer. Also shown are renal nerves 208 that lie within the adventitia 204, on the surface of the renal artery 104, and adjacent to the renal artery 104. The renal nerves 208 surround the renal artery 104. Different individuals have renal nerves 208 in different locations around the renal artery 104. Thus, the renal nerves 208 may be at different radial distances R from the central axis A of the renal artery 104, and also may be at different locations around the circumference of the renal artery 104. It is not practical to locate the renal nerves by referring to anatomical landmarks. Moreover, it is difficult or impossible to locate individual renal nerves 208 using common in vivo imaging technology.

As explained above in the Background, renal nerve ablation (aka renal denervation) can be used to treat hypertension. Various studies have confirmed that renal nerve activity has been associated with hypertension, and that ablation of the renal nerves can improve renal function and reduce hypertension. In a typical procedure, a catheter (aka probe) is introduced into a hypertensive patient’s arterial vascular system and advanced into the renal artery 104. In a renal denervation procedure, renal nerves 208 located in the arterial wall and in regions adjacent to the renal artery 104 are ablated by a destructive means, such as radio frequency (RF) energy, microwave energy, ultrasound energy, pulsed electric field energy, cryotherapy, laser or chemical agents to limit the renal nerve activity, thereby reducing hypertension in the patient. The destructive means that is used to perform such a renal denervation procedure can be a transducer that is located on a distal portion of a catheter that is inserted into the renal artery 104. For much of the remaining discussion, it will be assumed that the transducer is an ultrasound transducer that can be activated to deliver unfocused ultrasonic energy radially outwardly so as to suitably heat, and thus treat, tissue within the target anatomical region surrounding the renal artery 104. Such a transducer can be activated at a frequency, duration, and energy level suitable for treating the targeted tissue. In one non-limiting example, the unfocused ultrasonic energy generated by the transducer may target select nerve tissue of the subject, and may heat such tissue in such a manner as to neuromodulate (e.g., fully or partially ablate, necrose, or stimulate) the nerve tissue. One of skill in the art will recognize that other mechanisms may be used to denervate the nerve tissue. Non-limiting examples of other mechanisms used to perform a renal denervation procedure include RF energy, microwave energy, pulsed electric field energy, chemical agents, optical energy, laser and/or cryotherapy, but is not limited thereto.

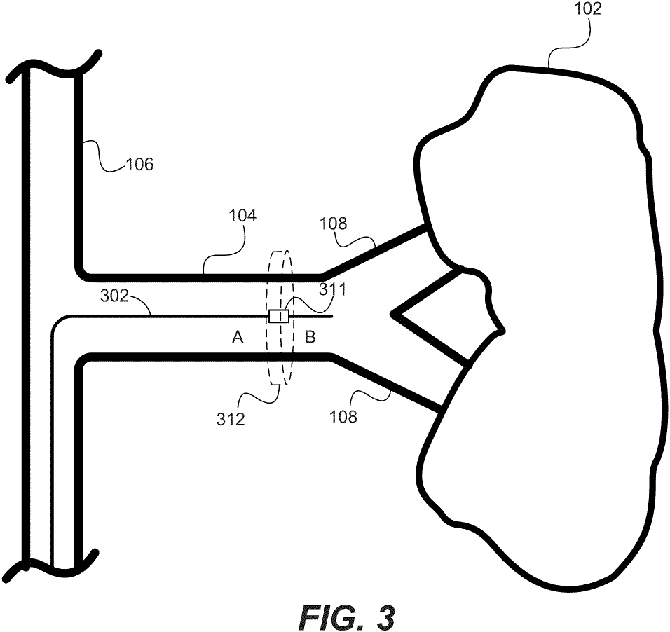

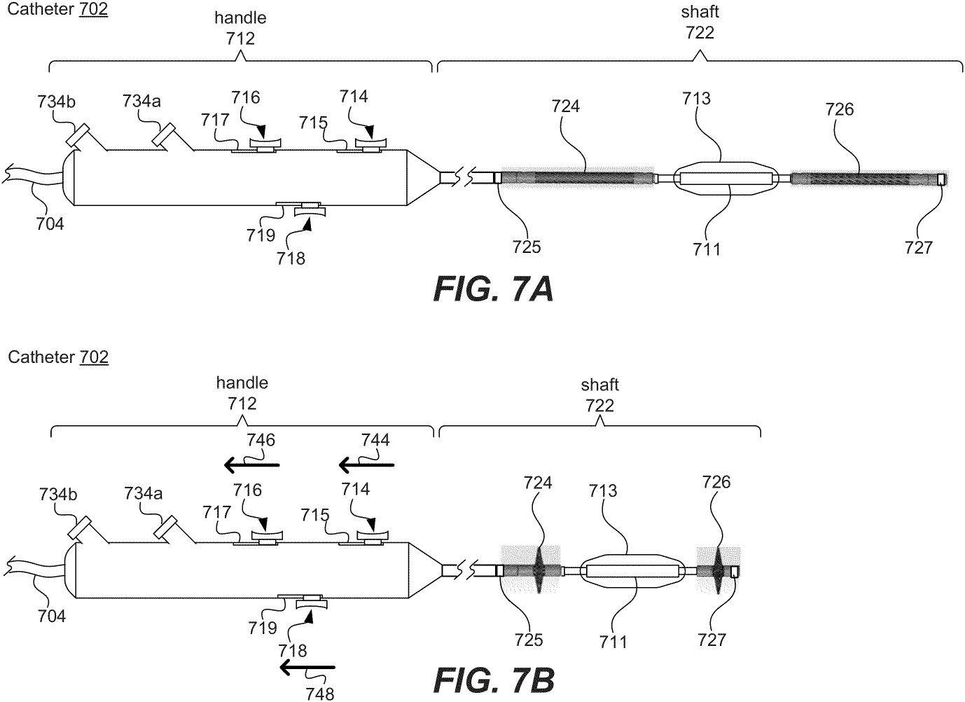

Reference is now made to FIG. 3, which is similar to FIG. 1 in that it shows a kidney 102 that receives blood via the abdominal aorta 106, a renal artery 104, and branch vessels 108, which are labeled the same in FIG. 3 as they were in FIG. 1. FIG. 3 also shows a catheter 302 having a transducer 311 located on a distal portion of the catheter 302, wherein the distal portion of the catheter 302, which includes the transducer 311, has been inserted into the renal artery 104. While not shown in FIG. 3, the transducer 311 may (or may not) be located within a balloon, example details of which are described below with reference to FIGS. 7A and 7B. In FIG. 3, the dashed cylindrical region labeled 312 illustrates an example denervation treatment region, wherein the portion of the renal artery 104 labeled A is proximal or upstream relative to the treatment region 312, and the portion of the renal artery labeled B is distal or downstream relative to the treatment region 312. An actual denervation treatment region may differ from the dashed cylindrical region labeled 312.

A denervation procedure can be performed by performing ablation therapy using the transducer 311 to emit ultrasound ablation energy into the treatment region 312. Alternatively, or additionally, some other means for performing the ablation therapy of the denervation procedure may be used, such as, but not limited to, emitting RF energy, pulsed electric field energy, or microwave ablative energy. Alternatively, or additionally, other means for providing ablation therapy may be used to perform the denervation procedure, such as chemical agents, optical, laser and/or cryotherapy. Following such a denervation procedure, it would be beneficial to determine the efficacy of the denervation procedure, in order to determine whether the denervation procedure was successful and can thus be terminated, or to determine whether further ablation therapy should be delivered, e.g., because the denervation procedure was unsuccessful or incomplete.

Prior to performing such a denervation procedure, pre-denervation neural activity (aka baseline neural activity) could be sensed using the same catheter 302, or a separate catheter, so that the pre-denervation neural activity can be compared to post-denervation neural activity, for the purpose of determining whether or not the denervation procedure was successful, and thus, for determining whether the denervation procedure can be terminated, or whether addition ablation energy (or other type of ablation therapy) should be delivered. Further, even if pre-denervation neural activity (aka baseline neural activity) is not sensed, it can still be useful to sense post-denervation neural activity, for the purpose of determining whether the denervation procedure was successful, and thus, can be terminated, or whether addition ablation energy (or other type of ablation therapy) should be delivered. The neural activity could be sensed using electrodes of a catheter, which can be the same catheter 302, or a separate catheter. Such electrodes can be positioned within the treatment region 312, upstream the treatment region 312, or downstream the treatment region 312. The neural activity that is sensed using such electrodes could be native neural activity, which can alternatively be referred to as spontaneous neural activity. Alternatively, or additionally, the neural activity that is sensed using such electrodes can be an evoked neural response, which is responsive to stimulation energy delivered using further electrodes of the catheter that is inserted into the renal artery 104.

Following ablation energy (or other type of ablation therapy) being delivered to the treatment region 312, if one or more electrodes used to sense neural activity are positioned within the region A in FIG. 3 that is proximal (i.e., upstream of) the treatment region 312 within the renal artery 104, then it may be difficult to determine the efficacy of the denervation procedure because even if the renal nerves of interest within the treatment region 312 were sufficiently destroyed, neural activity of efferent renal nerves attempting to carry neural impulses from the brain toward the kidney 102 may be detected by the electrode(s) that are proximal (i.e., upstream of) the treatment region 312. If the one or more electrodes used to sense neural activity are instead positioned within the region B in FIG. 3 that is distal (i.e., downstream) of the treatment region 312 within the renal artery 104, then it may also be difficult to determine the efficacy of the denervation procedure because even if the renal nerves of interest within the treatment region 312 were sufficiently destroyed, neural activity of afferent renal nerves attempting to carry neural impulses from the kidney 102 toward the brain may be detected by the electrode(s) that are distal (i.e., downstream) of the treatment region 312. More generally, the discussion of FIG. 3 is used to explain why, when there is a desire to destroy the nerves within a specific length or segment of a biological lumen (e.g., a renal artery) as part of a denervation procedure, it is often difficult to know when the nerve destruction has been sufficient such that the denervation procedure can be considered complete.

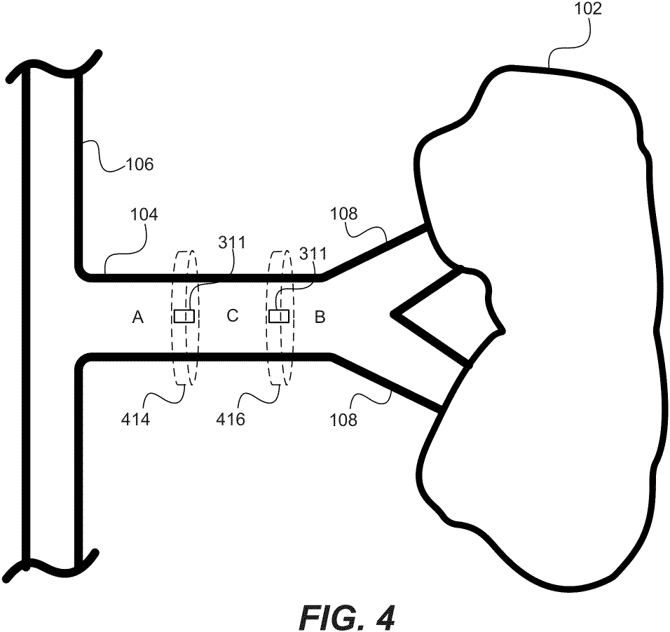

As will now be explained with reference to FIG. 4, in accordance with certain embodiments of the present technology, in order to improve the ability to determine the efficacy of a denervation procedure, nerve destruction is performed at two different longitudinally spaced apart locations along a biological lumen (e.g., a renal artery) and electrodes are used to sense for a native or evoked neural response at a longitudinal location between the two nerve destruction sites.

More specifically, in accordance with certain embodiments of the present technology, when there is a desire to destroy the nerves within a specific length or segment of a biological lumen (e.g., a renal artery), a catheter (e.g., 302 in FIG. 3) is used to deliver ablation energy (or another type of ablation therapy) at a first longitudinal location (e.g., at the distal end of the specific length or segment of the biological lumen) and then used to deliver ablation energy (or another type of ablation therapy) at a second longitudinal location (e.g., at the proximal end of the specific length or segment of the biological lumen), or vice versa. Thereafter, the same catheter, or a separate catheter, is used to sense a native and/or evoked neural response between the first and the second longitudinal locations of the biological lumen (i.e., between the two nerve destruction sites) to determine if sufficient nerve destruction was performed. It can be determined that sufficient nerve destruction has been performed if the native and/or evoked neural response is below a respective specified threshold. On the other hand, it can be determined that sufficient nerve destruction has not been performed if the native and/or evoked neural response exceeds the respective specified threshold. If sufficient nerve destruction has not been performed, then additional ablation energy (or another type of ablation therapy) can be applied at the first and/or second longitudinal locations. Additionally, or alternatively, additional ablation energy (or another type of ablation therapy) can be delivered at one or more longitudinal locations between the aforementioned first and second longitudinal locations. Additionally, or alternatively, additional ablation energy (or another type of ablation therapy) can be delivered at one or more longitudinal locations proximal (upstream of) the aforementioned first and second longitudinal locations, and/or at one or more longitudinal locations distal (downstream of) the aforementioned first and second longitudinal locations.

In the description herein, a denervation procedure is often described as being performed by delivering ablation energy, such as by using an appropriate transducer of a catheter to deliver ultrasound energy, RF energy, pulsed electric field energy or microwave energy. However, it should also be appreciated from the description herein that the use of other types ablation therapy can alternatively or additionally be used while being within the scope of the embodiments described herein. Such other types of ablation therapy include, but are not limited to, cryotherapy, chemicals (e.g., drugs or other agents), laser light or optical energy, magnetic energy, direct heat energy, radiation (e.g., infrared, visible, gamma), or combinations thereof.

Referring now to FIG. 4, the dashed cylindrical region labeled 414 illustrates an example first denervation treatment region, and the dashed cylindrical region labeled 416 illustrates an example second denervation treatment region, wherein the treatment regions 414 and 416 are longitudinally spaced apart from one another along the renal artery 104. Still referring to FIG. 4, the portion of the renal artery 104 labeled A is proximal or upstream relative to both of the treatment regions 414 and 416, the portion of the renal artery 104 labeled B is distal or downstream relative to both of the treatment regions 414 and 416, and the portion of the renal artery 104 labeled C is between the treatment regions 414 and 416. In accordance with certain embodiments of the present technology, one or more sensing electrodes is/are positioned within the portion of the renal artery 104 labeled C (i.e., between the treatment regions 414 and 416) for the purpose of determining the efficacy of the ablation energy delivered to the treatment regions 414 and 416.

In such embodiments, detecting substantially no neural activity between the two ablation locations (i.e., between the treatment regions 414 and 416) is indicative of both the efferent and afferent traffic being cut. In other words, if the ablations performed at both of the treatment regions 414 and 416 are successful, substantially no neural activity should be detected between the treatment regions 414 and 416. More specifically, following ablation energy being delivered to the treatment regions 414 and 416, if the ablation energy successfully denervated the renal nerves at both of the treatment regions 414 and 416, then the ablation energy delivered to the treatment region 414 should prevent efferent renal nerve impulses (traveling from the brain toward the kidney 102) from reaching the region C, and the ablation energy delivered to the treatment region 416 should prevent afferent renal nerves impulses (traveling from the kidney 102 toward the brain) from reaching the region C. Accordingly, if the ablations performed at both of the treatment regions 414 and 416 are successful, then one or more electrodes positioned within the region C (in FIG. 4) of the renal artery 104 should sense substantially no renal nerve activity, i.e., sensed renal nerve activity should be below a specified threshold.

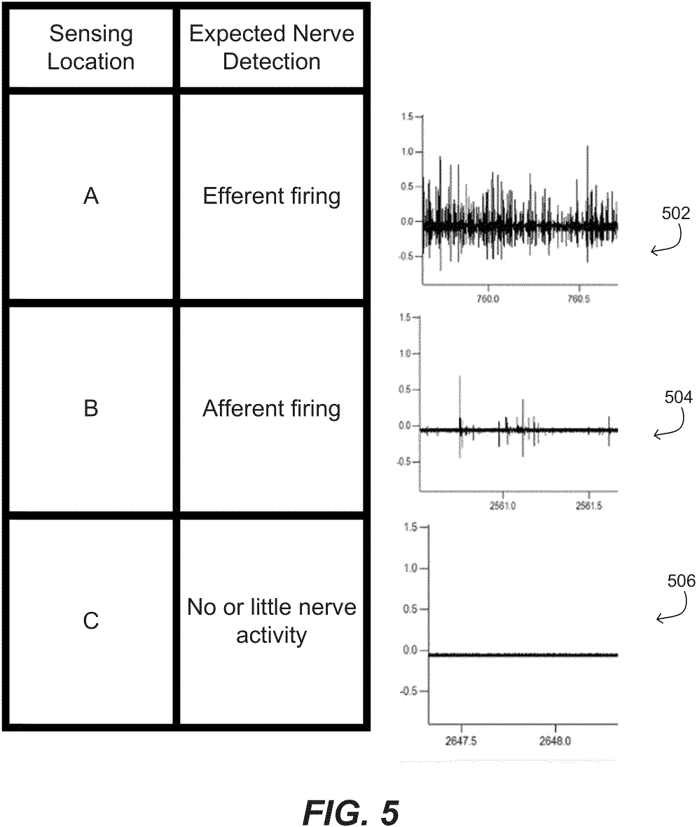

FIG. 5 illustrates expected sensed nerve activity at the three regions, labeled A, B, and C in FIG. 4, following the denervation procedure described above with reference to FIG. 4. Referring to FIG. 5, the graph 502 illustrates expected neural activity that may be sensed at the region A in FIG. 4, which is proximal (i.e., upstream of) both of the treatment regions 414 and 416, and thus, is indicative of efferent renal nerves firing. The graph 504 illustrates expected neural activity that may be sensed at the region B in FIG. 4, which is distal (i.e., downstream of) both of the treatment regions 414 and 416, and thus, in indicative of afferent renal nerves firing. Still referring to FIG. 5, the graph 506 illustrates expected neural activity that may be sensed at the region C, which is longitudinally between the treatment regions 414 and 416, if the ablation energy delivered to the respective treatment regions 414 and 416 successfully ablated the renal nerves therein. Accordingly, the graph 506 shows substantially no neural activity being sensed within the region C.

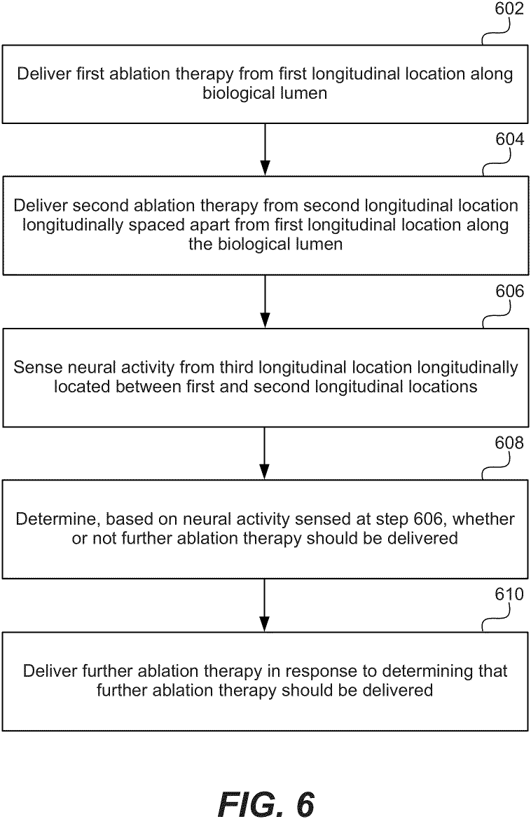

The high level flow diagram of FIG. 6 will now be used to describe methods for performing a denervation procedure and determining an efficacy thereof, which can be implemented using a system (e.g., 800) which can include or be electrically coupled to a catheter (e.g., 302, 702) in accordance with certain embodiments of the present technology. Referring to FIG. 6 (and FIGS. 3, 4, 7 and 8), step 602 involves delivering first ablation therapy from a first longitudinal location along a biological lumen (e.g., a renal artery). Step 604 involves delivering second ablation therapy from a second longitudinal location longitudinally spaced apart from the first longitudinal location along the biological lumen. For example, referring briefly back to FIG. 4, step 602 can be performed by delivering the first ablation therapy from a transducer (e.g., 311, 711) longitudinally positioned within the renal artery 104 such that the ablation occurs within the treatment region 414; and step 604 can be performed by delivering the second ablation therapy from the transducer (e.g., 311, 711 or another transducer) longitudinally positioned within the renal artery 104 such that the ablation occurs within the treatment region 416. Alternatively, step 602 can be performed by delivering the first ablation therapy from a transducer (e.g., 311, 711) longitudinally positioned within the renal artery 104 such that the ablation occurs within the treatment region 416; and step 604 can be performed by delivering the second ablation therapy from the transducer (e.g., 311, 711 or another transducer) longitudinally positioned within the renal artery 104 such that the ablation occurs within the treatment region 414. In other words, the first ablation therapy can be delivered more proximal (downstream) than the second ablation therapy, or vice versa, depending upon the specific implementation. In the above examples, the transducer (e.g., 311, 711) is positioned at a first location within the biological lumen (e.g., renal artery 104) and then step 602 is performed, and thereafter the transducer (e.g., 311, 711) is repositioned to be at a second location within the biological lumen (e.g., renal artery 104) that is either upstream or downstream of the first location. Alternatively, two transducers longitudinally spaced apart from each other could be used to delivery ablation therapy at the first and second locations within the biological lumen (e.g., renal artery 104).

In accordance with certain embodiments, a same transducer (e.g., 311, 711) is used to perform both of steps 602 and 604 by initially maneuvering a catheter (e.g., 302, 702) including the transducer (e.g., 311, 711) such that the transducer (e.g., 311, 711) is positioned at the first location, and performing step 602 while the transducer 311, 711 is positioned at the first location, and then maneuvering the catheter (e.g., 302, 702) including the transducer (e.g., 311, 711) such that the transducer (e.g., 311, 711) is positioned at the second location, and performing step 604 while the transducer (e.g., 311, 711) is positioned at the second location. More generally, a same therapy delivery element (e.g., 311, 711) can be used to perform both of steps 602 and 604 by initially maneuvering a catheter (e.g., 302, 702) including the therapy delivery element (e.g., 311, 711) such that the therapy delivery element (e.g., 311, 711) is positioned at the first location, and performing step 602 while the therapy delivery element (e.g., 311, 711) is positioned at the first location, and then maneuvering the catheter (e.g., 302, 702) including the therapy delivery element (e.g., 311, 711) such that the therapy delivery element (e.g., 311, 711) is positioned at the second location, and performing step 604 while the therapy delivery element (e.g., 311, 711) is positioned at the second location.

In others embodiment, a catheter (e.g., 302, 702) includes two transducers (or other type of therapy delivery elements) that are longitudinally spaced apart from one another along a shaft (e.g., 722) of the catheter (e.g., 302, 702), thereby enabling steps 602 and 604 to be performed without needing to reposition the catheter (e.g., 302, 702) between steps 602 and 604. More specifically, the catheter (e.g., 302, 702) that includes two transducers (or other type of therapy delivery elements) is maneuvered such that one of the transducers (or other type of therapy delivery element) is positioned at the first location, and the other one of the transducers (or other type of therapy delivery element) is positioned at the second location. Steps 602 and 604 can then be performed simultaneously by energizing the two transducers at the same time. Alternatively step 602 can be initially performing by initially energizing the one of the transducers positioned at the first location, and then step 604 can be performed by energizing the other one of the transducers that is positioned at the second location, or vice versa. An example of a catheter including two longitudinally spaced apart transducers is disclosed in U.S. Pat. Publication No. 2023/0021354, which is incorporated herein by reference. The use of other catheters including two longitudinally spaced apart transducers (or other type of therapy delivery elements) is also possible and within the scope of the embodiments described herein. The two transducers may or may not be located within one or more balloons (e.g., 713). More specifically, the two transducers can both be located within a same balloon (e.g., 713), in respective separate balloons (e.g., 713), or not located within any balloons. Examples of other types of therapy deliver elements that can be longitudinally spaced apart from one another along a shaft (e.g., 722) of a catheter (e.g., 302, 702), and can be used to perform steps 602 and 604, include cryotherapy delivery elements, chemical deliver elements, and lasers, but are not limited thereto. The therapy delivery elements described above can also be referred to herein as ablation mechanisms. Such therapy delivery elements can also be referred to herein as an element configured to selectively deliver ablation therapy.

In accordance with certain embodiments, the ablation therapy delivered at an instance of step 602 can involve performing multiple ablations from the first longitudinal location. For example, multiple circumferential ablations can be performed from the same first longitudinal location, at the same time, or one after the other in a time multiplexed manner. This can be achieved, for example, by performing multiple separate focal ablations from the first longitudinal location. For a more specific example, multiple (e.g., four) RF electrodes can be circumferentially distributed about a circumference of a portion of a catheter (e.g., 302, 702). Such multiple RF electrodes can be used to emit ablation energy from a same first longitudinal location, in different radial directions, simultaneously, or in a time multiplexed manner. Similarly, ablation therapy delivered at an instance of step 604 can involve performing multiple ablations from the second longitudinal location. Additionally, the further ablation performed at an instance step 608, which is discussed below, can similarly involve performing multiple ablations from a same longitudinal location.

Referring again to FIG. 6, step 606 involves sensing neural activity from a third longitudinal location that may be longitudinally located between the first and the second longitudinal locations, from which the first and the second ablation therapy were respectively delivered (at steps 602 and 604), to thereby quantify to what extent nerves surrounding the biological lumen and adjacent to at least one of the first and the second longitudinal locations were affected by the delivering of the first and the second ablation therapy. For example, referring briefly back to FIG. 4 again, the sensing at step 606 can be performed from a location within the region C shown in FIG. 4. Additional details of how step 606 can be performed are provided below.

Referring again to FIG. 6, optional but preferred step 608 involves determining, based on the sensed neural activity (sensed at step 606), whether or not further ablation therapy should be delivered from a location along a longitudinal length of the biological lumen. More specifically, a measure of the neural activity sensed at step 606 can be compared to a corresponding threshold at step 608, and if the measure of the neural energy is less than the threshold there can be a determination at step 608 that the denervation procedure was successful, and that no further denervation of the biological lumen is needed. By contrast, if the measure of the neural energy exceeds the corresponding threshold there can be a determination at step 608 that the denervation procedure was not successful, and that further denervation of the biological lumen is still needed. If further denervation of the biological lumen is still needed, then at optional but preferred step 610 further ablation therapy can be delivered at the first longitudinal location referred to at step 602, at the second longitudinal location referred to at step 604, and/or between the first and the second longitudinal locations. Alternatively, or additionally, further ablation therapy may be delivered at step 610 proximal to both the first and the second longitudinal locations, and/or distal to both the first and the second longitudinal locations. The measure of neural activity sensed at step 606 can be, e.g., a maximum peak amplitude of the sensed neural activity. Alternatively, where there are multiple peaks in a sensed signal indicative of neural activity, an average or median amplitude can be determined, and that average or median amplitude can be the measure of neural activity that is compared to a threshold. Alternatively, a curve can be fit to a portion of the sensed signal indicative of neural activity, and an area under the curve can be the measure of neural activity that is compared to a threshold. Other variations are also possible and within the scope of the embodiments described herein. The specific threshold to which a measure of neural activity is compared will depend on how the specific measure of neural activity is determined.

In accordance with certain embodiments, step 602 is performing using a transducer (e.g., 311, 711) located on a distal portion of a catheter (e.g., 302, 711) that is inserted into the biological lumen such that the transducer (e.g., 311, 711) is positioned at the first longitudinal location, and step 604 is performing using the transducer (e.g., 311, 711) of the distal portion of the catheter (e.g., 302, 711) that is inserted into the biological lumen, after the transducer (e.g., 311, 711) is moved from being positioned at the first longitudinal location to being positioned at the second longitudinal location. In accordance with certain embodiments, the transducer (e.g., 311, 711) is an ultrasound transducer, in which case, step 602 can be performed by emitting first ultrasound energy from the ultrasound transducer (e.g., 311, 711) while the ultrasound transducer (e.g., 311, 711) is positioned at the first longitudinal location, and step 604 can be performed by emitting second ultrasound energy from the ultrasound transducer (e.g., 311, 711) while the ultrasound transducer (e.g., 311, 711) is positioned at the second longitudinal location. In accordance with other embodiments, step 602 can be performed by emitting first RF energy using one or more electrodes positioned at the first longitudinal location, and step 604 can be performed by emitting second RF energy using one or more electrodes positioned at the second longitudinal location. As noted above, other types of ablation mechanisms can be used to perform steps 602 and 604, such as, but not limited to microwave energy, cryotherapy, chemical agents, optical energy, laser and/or pulsed electric field ablation.

In accordance with certain embodiments, step 606 is performed using an electrode (e.g., 726, 727) positioned at the third longitudinal location that may be longitudinally located between the first and the second longitudinal locations. Such an electrode (e.g., 726, 727), which is used for the sensing neural activity from the third longitudinal location, can be included on a distal portion of a catheter (e.g., 302, 702), which can be a same catheter as or a different catheter than the catheter that includes the transducer (e.g., 311, 711) and/or other ablative means.

In accordance with certain embodiments, the neural activity that is sensed from the third longitudinal location at step 606 is native (aka spontaneous) neural activity. In accordance with other embodiments, the neural activity that is sensed from the third longitudinal location comprises an evoked neural response to stimulation that is delivered. Such stimulation to evoke a neural response can be delivered from at least one electrode (e.g., 726, 727) positioned within the biological lumen proximal (upstream of) both the first and the second longitudinal locations, or from at least one electrode (e.g., 726, 727) that is positioned within the biological lumen distal (downstream of) both the first and the second longitudinal locations. Regardless of whether the neural activity that is sensed at step 606 is native (aka spontaneous) neural activity, or evoked neural activity, the neural activity that is sensed at step 606 can be sensed by an electrode (e.g., 726, 727) positioned between the first and the second longitudinal locations, along with a further electrode. In certain embodiments, the further electrode is also positioned within the biological lumen between the first and the second longitudinal locations, in which case both of the electrodes (e.g., 726, 727) used to sense the native (aka spontaneous) neural activity are positioned between the first and the second longitudinal locations. For example, referring briefly back to FIG. 4, assuming ablation therapy is delivered to each of the treatment regions 414 and 416, in certain embodiments a pair of electrodes (e.g., 726, 727) that are both positioned within the region C (between the treatment regions 414 and 416) are used to sense native and/or evoked neural activity so that the efficacy of the denervation procedure can be determined based thereon at step 608. The electrodes (e.g., 726, 727) that are used for sensing neural activity can be referred to herein as sensing electrodes (e.g., 726, 727). In the embodiment just described above, the two sensing electrodes (e.g., 726, 727) were both described as being positioned within the biological lumen (e.g., a renal artery) between the first and the second longitudinal locations at which the first and the second ablation therapy were delivered respectively at steps 602 and 604.

In an alternative embodiment, a first sensing electrode is positioned within the biological lumen (e.g., a renal artery) between the first and the second longitudinal locations at which the first and the second ablation therapy were delivered respectively at steps 602 and 604, while a second (aka further) sensing electrode is an external skin electrode.

In another alternative embodiment, a first sensing electrode is positioned within the biological lumen (e.g., a renal artery) between the first and the second longitudinal locations at which the first and the second ablation therapy were delivered respectively at steps 602 and 604, while a second (aka further) sensing electrode is located proximal (i.e., upstream of) where the first and the second longitudinal locations at which ablation therapy was delivered at steps 602 and 604. For example, referring briefly back to FIG. 4, assuming ablation therapy is delivered to each of the treatment regions 414 and 416, in certain embodiments a first sensing electrode is positioned within the region C (between the treatment regions 414 and 416) and a second sensing electrode is positioned within the region A (proximal or upstream of both of the treatment regions). Both the first and the second sensing electrodes can be located on a catheter that is used to sense neural activity, which catheter may or may not be the same catheter that includes a transducer (e.g., 311) and/or other ablative means and that is used to deliver the ablation therapy at steps 602 and 604. Where the first and the second sensing electrodes are located on a different catheter than the catheter used to deliver the ablation therapy at steps 602 and 604, the catheter used to perform steps 602 and 604 is removed following step 604, and the different catheter is inserted into the biological lumen between steps 604 and 606. In other words, catheters can be swapped out and in between steps 604 and 606. In certain embodiments, a first sensing electrode is located on a catheter (which may or may not be the same catheter that includes a transducer, e.g., 311, and that is used to deliver the ablation therapy at steps 602 and 604) and is positioned within the biological lumen (e.g., a renal artery) between the first and the second longitudinal locations at which ablation therapy was delivered at steps 602 and 604, while a second sensing electrode is located on a distal end of an introducer sheath that is used for inserting the catheter that includes the first sensing electrode into the biological lumen (e.g., a renal artery).

In still another alternative embodiment, a first sensing electrode is positioned within the biological lumen (e.g., a renal artery) between the first and the second longitudinal locations at which ablation therapy was delivered at steps 602 and 604, while a second (aka further) sensing electrode is located distal (i.e., downstream of) the first and the second longitudinal locations at which ablation therapy was delivered at steps 602 and 604. For example, referring briefly back to FIG. 4, assuming ablation therapy is delivered to each of the treatment regions 414 and 416, in certain embodiments a first sensing electrode is positioned within the region C (between the treatment regions 414 and 416) and a second sensing electrode is positioned within the region B (distal or downstream of both of the treatment regions). Both the first and the second sensing electrodes can both be located on a same catheter, which may or may not be the same catheter that includes a transducer (e.g., 311) or other ablation means and that is used to deliver the ablation therapy at steps 602 and 604. It is also possible that the second sensing electrode can be separate from a catheter, e.g., be located on the distal end of a guide wire that is used for guiding the catheter that includes the first sensing electrode into the biological lumen (e.g., a renal artery).

As noted above, in certain embodiments the neural activity that is sensed at step 606 can be an evoked neural response to stimulation energy that is delivered using one or more electrodes (e.g., 724, 725) of a catheter (e.g., 302, 702) that is inserted into the biological lumen, which catheter (e.g., 302, 702) also includes at least one of the sensing electrodes (e.g., 726, 727) used at step 606, and which catheter (e.g., 302, 702) may or may not be the same catheter used to deliver the ablation therapy at steps 602 and 604, depending upon the specific implementation. The pair of electrodes (e.g., 724, 725) that are used to deliver the stimulation that evokes a neural response can be referred to herein as first and second stimulation electrodes (e.g., 724, 725). In certain embodiments, both the first and the second stimulation electrodes (e.g., 724, 725) are positioned within the biological lumen (e.g., a renal artery 104) between the first and the second longitudinal locations at which ablation therapy was delivered at steps 602 and 604. For example, referring briefly back to FIG. 4, assuming ablation therapy is delivered to each of the treatment regions 414 and 416, both the first and the second stimulation electrodes (e.g., 724, 725) can be positioned within the region C (between the treatment regions 414 and 416).

In other embodiments, only the first stimulation electrode is positioned within the biological lumen (e.g., a renal artery 104) between the first and the second longitudinal locations at which ablation therapy was delivered at steps 602 and 604, while the second stimulation electrode is located on the same catheter as the first stimulation electrode but is located either proximal (aka upstream of) both the first and the second longitudinal locations, or distal (aka downstream of) both the first and the second longitudinal locations. For example, referring briefly back to FIG. 4, assuming ablation therapy is delivered to each of the treatment regions 414 and 416, in certain embodiments a first stimulation electrode is positioned within the region C (between the treatment regions 414 and 416) and a second stimulation electrode is positioned within the region A (proximal or upstream both of the treatment regions) or within the region B (distal or downstream of both of the treatment regions). One of the first and the second stimulation electrodes should be configured as the stimulation anode, while the other is configured as the stimulation cathode.

In still other embodiments, the second stimulation electrode (that is used as the return electrode), rather than being located on the same catheter as the first stimulation electrode, is located on a distal end of an introducer sheath that is used to insert the catheter that includes the first stimulation electrode into the biological lumen, or is located on a distal end of a guidewire that is used to guide the catheter that includes the first stimulation electrode into the biological lumen. In another embodiment, the second stimulation electrode (that is used as the return electrode), rather than being located on the same catheter as the first stimulation electrode, is an external skin electrode.

Alternatively, stimulation can be delivered proximal the most proximal ablation site and the evoked response neural activity can be sensed distal the most distal ablation site. In other words, stimulation to evoke a neural response can be delivered via one or more stimulation electrodes located proximal (i.e., upstream of) both the first and the second longitudinal locations, and can be sensed using one or more sensing electrodes located distal (i.e., downstream of) both the first and the second longitudinal locations. For example, referring briefly back to FIG. 4, assuming ablation therapy is delivered to each of the treatment regions 414 and 416, in certain embodiments stimulation can be delivered from one or more electrodes within the region A, and an evoked neural response can be sensed using one or more sensing electrodes within the region B.

Another alternative is to deliver stimulation distal to the most distal ablation site and to sense the evoked neural response proximal to the most proximal ablation site. In other words, stimulation to evoke a neural response can be delivered via one or more stimulation electrodes located distal (i.e., downstream of) both the first and the second longitudinal locations, and can be sensed using one or more sensing electrodes located proximal (i.e., upstream of) both the first and the second longitudinal locations. For example, referring briefly back to FIG. 4, assuming ablation therapy is delivered to each of the treatment regions 414 and 416, in certain embodiments stimulation can be delivered from one or more electrodes within the region B, and an evoked neural response can be sensed using one or more sensing electrodes within the region A.

As was noted above, the same catheter (e.g., 302, 702) that was used to perform steps 602 and step 604 can also be used to perform step 606, regardless of whether the neural activity sensed at step 606 is native (aka spontaneous) neural activity or an evoked response to stimulation delivered via one or more electrodes (e.g., 724, 725) of the catheter (e.g., 302, 702). Alternatively, as was noted above, a different catheter than was used to perform steps 602 and step 604 can be used to perform step 606, regardless of whether the neural activity sensed at step 606 is native (aka spontaneous) neural activity or an evoked response to stimulation delivered via the different catheter, in which case catheters should be swapped between steps 604 and 606.

Embodiments of the present technology can be implemented using various different catheter implementations, and thus, are not limited to use with any specific catheter and or system of which a catheter is a part. Nevertheless, for completeness, an example catheter and system that can be used to implement embodiments of the present technology are described below. More specifically, FIGS. 7A through 11B are used to describe an example catheter and system that can be used to implement embodiments of the present technology that were described above. Such a system can also be referred to as an apparatus herein.

### EXAMPLE CATHETER

FIG. 7A shows a catheter 702 with its selectively deployable electrodes 724 and 726 in their non-deployed positions. The catheter 702 includes a catheter handle 712 and a catheter shaft 722. In addition to including the selectively deployable electrodes 724 and 726, the catheter shaft 722 is also shown as including a non-deployable electrode 725 that is proximal to the selectively deployable electrode 724, and a non-deployable electrode 727 that is distal the selectively deployable electrode 726. The selectively deployable electrode 724 can also be referred to as the proximal selectively deployable electrode 724, or more succinctly as the proximal electrode 724, or even more succinctly as the electrode 724. The selectively deployable electrode 726 can also be referred to as the distal selectively deployable electrode 726, or more succinctly as the distal electrode 726, or even more succinctly as the electrode 726. The catheter shaft 722 can also be referred to more succinctly herein as the shaft 722. The catheter 702 can be a specific implementation of the catheter 302 shown in and discussed above with reference to FIG. 3.

The catheter 702 is also shown as including a balloon 713 positioned longitudinally between the electrodes 724 and 726, wherein the balloon 713 is selectively inflatable and deflatable. The balloon 713 can also be referred as a selectively inflatable balloon 713, a selectively deployable balloon 713, or more succinctly as a balloon 713. When the balloon 713 is deflated, it can also be referred to as being non-inflated or in its non-deployed position. When the balloon 713 is inflated, it can also be referred to as being in its deployed position. As will be described in additional detail below, the balloon 713 can be selectively inflated by injecting a fluid into the balloon 713, and the balloon 713 can be selectively deflated by removing the fluid from the balloon 713. The balloon 713 can be made of an electrically insulating material such as polyamide, polyethylene terephthalate, or thermoplastic elastomer. In specific embodiments the balloon 713 is made from nylon, a polyimide film, a thermoplastic elastomer (such as those marked under the trademark PEBAX™), a medical-grade thermoplastic polyurethane elastomers (such as those marketed under the trademark PELLETHANE™), pellethane, isothane, or other suitable polymers or any combination thereof, but is not limited thereto.

The catheter handle 712, which can also be referred to more succinctly as the handle 712, includes actuators 714, 716, and 718, which can be used to selectively deploy the electrodes 724, 726, as well as to adjust a longitudinal distance between the electrodes 724, 726, as will be described in additional detail below. The actuators 714, 716, and 718 are respectively slidable within slots 715, 717, and 719 in the handle 712, and thus, the actuators 714, 716, and 718 can also be referred to as sliders. The catheter handle 712 is also shown as including a fluidic inlet port 734a and a fluidic outlet port 734b.

A fluid (e.g., expelled from a pressure syringe) can enter a fluid lumen (in the catheter shaft 722), via the fluidic inlet port 734a of the catheter 702, and then enter and at least partially fill the balloon 713. Fluid can be drawn from the balloon 713 (e.g., using a vacuum syringe) through another fluid lumen (in the catheter shaft 722) and out the fluidic outlet port 734b of the catheter 702. In this manner, the fluid can be used to selectively inflate and selectively deflate the balloon 713. In certain embodiments, fluid can be simultaneously injected into and removed from the balloon 713 to thereby circulate the fluid through the balloon 713.

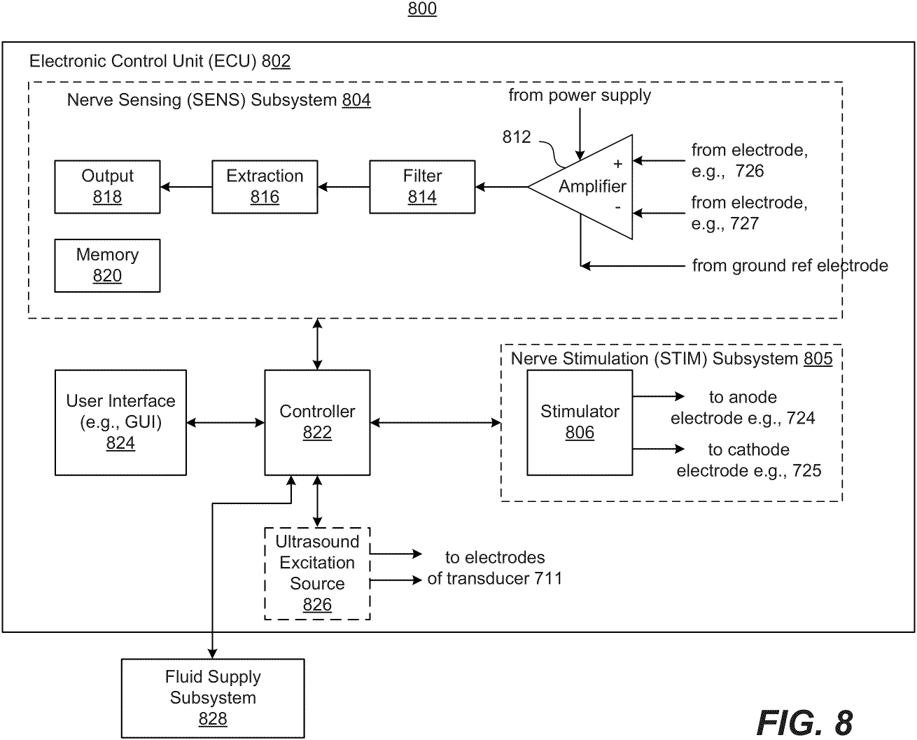

The catheter 702 can also be referred to as an intraluminal microneurography probe 702, or more succinctly, as a probe 702. A cable 704, which extends from a proximal portion of the handle 712, provides for electrical connections between the catheter 702 (and more specifically, the electrodes thereof) and an electrical control unit (ECU), an example of which is described below with reference to FIG. 8.

Still referring to FIG. 7A, a transducer 711 is shown as being within a balloon 713. The transducer 711 is an example of an ablation element (aka an element configured to selectively deliver ablation therapy) that is included on the shaft 722 and configured to ablate nerve tissue using ultrasound energy. In other embodiments, the transducer and balloon may be replaced by a helical structure carrying a plurality of electrodes configured to deliver RF and/or pulsed electric field RF energy. In other embodiments, the transducer and balloon may be replaced by a microwave transmitting element, which may or may not be within an expandable centering element. In other embodiments, the transducer may be replaced by a cryotherapeutic applicator. In still other embodiments, the transducer and balloon may be replaced by an infusion needle configured to deliver an ablative chemical to the renal nerves. It is also possible that the shaft 722 includes two longitudinally spaced apart transducers, e.g., as disclosed in U.S. Pat. Publication No. 2023/0021354, which was incorporated herein by reference above.

Where a transducer 711 is within the balloon 713, the fluid that is circulated through the balloon 713 can be referred to as a cooling fluid that is used to cool the transducer 711 and/or to cool a portion of a biological lumen that the balloon 713 is within, and/or to cool the biological tissue surrounding the lumen. It is also possible that the catheter 702 is devoid of the transducer 711 or other ablative means, and that a separate catheter that includes a transducer or other ablative means is used to deliver ablation therapy (e.g., at step 602 and 604 in FIG. 6). Where the catheter 702 is devoid of the transducer 711 or other ablative means, one or more electrodes (e.g., 726, 727) of the catheter 702 can be used for sensing native neural activity. One or more electrodes (e.g., 724, 725) of the catheter 702 can be used for delivering stimulation energy and one or more further electrodes (e.g., 726, 727) of the catheter 702 can be used for sensing an evoked neural response to the stimulation energy.

When the catheter 702 is inserted into a biological lumen, such as an artery, vein or other vasculature, it is the distal portion of the catheter 702 (and more specifically the shaft 722) that is inserted into the biological lumen, and the proximal end of the catheter 702 (and more specifically the handle 712) that is used to maneuver the catheter 702. In the embodiment shown in FIGS. 7A and 7B, the electrode 726 can also be referred to as a distal selectively deployable electrode 726 as noted above, since it located closer to the distal end of the catheter 702 than to the proximal end of the catheter 702; and the electrode 724 can also be referred to as a proximal selectively deployable electrode 724 as noted above, since it is located closer to the proximal end of the catheter 702 than to the distal end of the catheter 702. For similar reasons, the electrode 725 can be referred to as the proximal non-deployable electrode 725, and the electrode 727 can be referred to as the distal non-deployable electrode 727.

FIG. 7B shows the catheter 702 with the electrodes 724 and 726 in their deployed (aka expanded) positions. In certain embodiments, the proximal selectively deployable electrode 724 is configured to be deployed (aka expanded) in response to the actuator 714 being slid in the proximal direction indicated by the arrow 744 in FIG. 7B. In such an embodiment, the proximal electrode 724 can be returned to its non-deployed (aka non-expanded or retracted) position in response to the actuator 714 being slid in the distal direction opposite the arrow 744 in FIG. 7B. More generally, the actuator 714 is used to selectively expand and retract the electrode 724.

In accordance with certain embodiments, the longitudinal distance between the distal electrode 726 and the proximal electrode 724 can be reduced by sliding the actuator 718 in the proximal direction indicated by the arrow 748 in FIG. 7B. Thereafter, the longitudinal distance between the distal electrode 726 and the proximal electrode 724 can be increased, if desired, by sliding the actuator 718 in the distal direction opposite the arrow 748 in FIG. 7B. More generally, the actuator 718 is used to adjust the longitudinal distance between the electrodes 724 and 726. The longitudinal distance between the proximal and distal electrodes 724, 726 can be any distance between the maximum and minimum longitudinal distance as controlled by a user using the actuator 718. In accordance with certain embodiments, the electrode 724 is configured to be deployed in response to the actuator 714 being slid in the proximal direction indicated by the arrow 744 in FIG. 7B. In accordance with certain embodiments, the distal electrode 726 is configured to be deployed in response to the actuator 716 being slid in the proximal direction indicated by the arrow 746 in FIG. 7B. In such an embodiment, the distal electrode 726 can be returned to its non-deployed position in response to the actuator 716 being slid in the distal direction opposite the arrow 746 in FIG. 7B. More generally, the actuator 716 is used to selectively expand and retract the electrode 726. Other variations are also possible and within the scope of the embodiment described herein.

Each of the selectively deployable electrodes 724, 726 can be made, for example, of a unitary nitinol tube that is laser cut to include apertures or openings having a predetermine pattern. In FIGS. 7A and 7B, each of the electrodes 724, 726 has laser cut spiral apertures that extend between proximal and distal portions of each of the electrodes 724, 726. The spiral apertures in each of the electrodes 724, 726 enable each of the electrodes to be selectively transitioned between their non-deployed and deployed positions. The apertures that are cut into the electrodes 724, 726 can have other shapes besides being spiral, so long as the apertures enable the electrodes to be transitioned between non-deployed and deployed positions. The selectively deployable electrodes 724, 726 can alternatively be mesh electrodes or spiral electrodes made of one or more electrically conductive wires, optionally with portions thereof being insulated. Other variations are also possible and within the scope of the embodiments described herein.

The catheter 702 can be configured to be introduced into a biological lumen, such as an artery, in a location near a body organ, such as a kidney. The catheter 702 can be introduced via an introducer sheath that is advanced to the intended catheter location in the biological lumen, and then withdrawn sufficiently to expose the shaft 722 to the biological lumen (e.g., renal artery 104). Once the shaft 722 is within the biological lumen, one of the electrodes 724, 726 can be deployed (aka expanded) using one of the actuators 714, 716 such that it is in contact with a portion of a circumferential interior wall of the biological lumen. The longitudinal distance between the electrodes 724 and 726 can then be adjusted, if desired, using the actuator 718. The other one of the electrodes 724, 726 can then be deployed (aka expanded) such that it is in contact with another portion of the circumferential interior wall of the biological lumen.

For example, where the catheter (e.g., 702) is inserted into a renal artery (e.g., 104) close to a kidney, the electrodes (e.g., 724, 726) can be positioned near a nerve bundle that connects the kidney to the central nervous system, as the nerve bundle tends to approximately follow the artery leading to most body organs. The nerve bundle tends to follow the artery more closely at the end of the artery closer to the kidney, while spreading somewhat as the artery expands away from the kidney. As a result, it is desired in some examples that the catheter shaft 722 is small enough to introduce relatively near the kidney or other organ, as nerve proximity to the artery is likely to be higher nearer the organ.

Once the catheter 702 is in place, a practitioner can use instrumentation (e.g., the ECU 802) coupled to the electrodes (e.g., 724, 725) to stimulate one or more nerves, and monitor for evoked nerve response signals used to characterize the nervous system response to certain stimulus. The transducer 711 and/or other ablative means are configured to ablate nerve tissue, such as by using ultrasound, RF, pulsed electric field RF, microwave, cryotherapy, or other energy or chemical means. Additionally, the catheter 702 can actively stimulate one or more nerves and sense resulting neural signals in between applications of ablation therapy via the transducer 711, enabling more accurate control of the degree and effects of nerve ablation. In other examples, a catheter 702 lacking a transducer or other ablative means can be removed via a sheath, and an ablation probe (aka catheter) inserted, with the ablation probe removed and the catheter 702 reinserted to verify and characterize the effects of the ablation probe.

Any one or more electrodes (e.g., 724, 725) of the catheter 702 can be selectively used to deliver stimulation energy to nerves surrounding a biological lumen. Similarly, any one or more electrodes (e.g., 726, 727) of the catheter 702 can be selectively used to sense neural activity of nerves surrounding a biological lumen, which can be spontaneous neural activity, or evoked neural activity.

For much of the below discussion, it is assumed that the transducer 711 is an ultrasound transducer that can be activated to deliver unfocused ultrasonic energy radially outwardly so as to suitably heat, and thus treat, tissue within the target anatomical region. The transducer 711 can be activated at a frequency, duration, and energy level suitable for treating the targeted tissue. In one nonlimiting example, the unfocused ultrasonic energy generated by the transducer 711 may target select nerve tissue of the subject, and may heat such tissue in such a manner as to neuromodulate (e.g., fully or partially ablate, necrose, or stimulate) the nerve tissue. The transducer 711 can be the same transducer 311 shown in FIGS. 3 and 4 and discussed above.

In accordance with certain embodiments, the transducer 711 includes a piezoelectric transducer body that comprises a hollow tube of piezoelectric material having an inner surface and an outer surface, with an inner electrode disposed on the inner surface of the hollow tube of piezoelectric material, and an outer electrode disposed on the outer surface of the hollow tube of piezoelectric material. In such embodiments, the hollow tube of piezoelectric material is an example of the piezoelectric transducer body. The hollow tube of piezoelectric material, or more generally the piezoelectric transducer body, can be cylindrically shaped and have a circular radial cross-section. However, in alternative embodiments the hollow tube of piezoelectric material can have other shapes besides being cylindrical with a circular radial cross-section. Other cross-sectional shapes for the hollow tube of piezoelectric material, and more generally the piezoelectric transducer body, include, but are not limited to, an oval or elliptical cross-section, a square or rectangular cross-section, pentagonal cross-section, a hexagonal cross-section, a heptagonal cross-section, an octagonal cross-section, and/or the like. The hollow tube of piezoelectric material, and more generally the piezoelectric transducer body, can be made from various different types of piezoelectric material, such as, but not limited to, lead zirconate titanate (PZT), polyvinylidene fluoride (PVDF), or other presently available or future developed piezoelectric ceramic materials. In other embodiments, the transducer 711 can be made of other materials and/or can have other shapes.

In certain embodiments, the transducer 711 is an ultrasound transducer configured to deliver acoustic energy in the frequency range of 8.5 to 9.5 MHz. In certain embodiments, the transducer is configured to deliver acoustic energy in the frequency range of 8.7-9.3 MHz or 8.695-9.304 MHz. Transducers delivering acoustic energy in the frequency range of 8.7-9.3 MHz have been shown to produce ablation up to mean depths of 6 mm. The piezoelectric transducer body is configured to generate ultrasonic waves in response to a voltage being applied between the inner and outer electrodes. One or both of the inner and outer electrodes can be covered by an electrical insulator to inhibit (and preferably prevent) a short circuit from occurring between the inner and outer electrodes when the ultrasound transducer is placed within an electrically conductive fluid and a voltage is applied between the inner and outer electrodes. Such an electrical insulator can be parylene, and more specifically, a parylene conformal coating, but is not limited thereto. An excitation source (e.g., 826 in FIG. 8) may be electrically coupled to inner and outer electrodes of the transducer 711, and may actuate the transducer 711 by applying a voltage between the inner and outer electrodes (or any other pair of electrodes), so as to cause the piezoelectric material of the piezoelectric transducer body to generate an unfocused ultrasonic wave that radiates radially outwardly.

### EXAMPLE ELECTRICAL CONTROL UNIT (ECU)

FIG. 8 is a high level block diagram of an electrical control unit (ECU) 802 that is configured to be in electrical communication with a catheter, such as the catheter 702 described above. The ECU 802, and the catheter (e.g., 702) to which the ECU 802 is electrically coupled via a cable (e.g., 704), can be referred to more generally as a system 800. The ECU 802 can process a received signal to produce an output signal, and present information including information about the output signal, the received signal, or processing information. Such a system 800 can be used, for example, in diagnostic procedures for assessing the status of a patient’s nervous activity proximate a biological lumen, such as a vein or an artery, e.g., a renal artery, or another type of blood vessel. Such a system 800 can be additionally, or alternatively, be used to select preferred denervation parameters for use in a denervation procedure. As will be described in additional details below, a same catheter (e.g., 702) that is used to assess the status of a patient’s nervous activity proximate and/or select preferred denervation parameters can also be used to perform a denervation procedure. Alternatively, it is possible that the catheter that is used to perform a denervation procedure differs from the catheter that is used to assess that status of a patient’s nervous activity proximate a biological lumen, in which case different catheters can be swapped in and out of a biological lumen during a procedure.

Still referring to FIG. 8, the ECU 802 includes a stimulator 806 electrically coupled to a selected pair of electrodes (e.g., 724, 725) of the catheter 702. The stimulator 806, which is part of a STIM circuit or subsystem 804, can selectively emit electrical signals (including stimulation pulses) having a specific voltage, amperage, duration, duty cycle and/or frequency of application that will cause nerve cell activation. For an example, the electrode 724 can be connected as the stimulation anode and the electrode 725 can be connected as the stimulation cathode, or vice versa. For another example, the electrode 725 can be connected as the stimulation anode and the electrode 726 can be connected as the stimulation cathode, or vice versa. Switches, which are not specifically shown, can be used to selectively control how various electrodes (e.g., 724, 725, 726 727) are coupled to various nodes of the ECU 802, such as to the input terminals of the amplifier 812, or to the output terminals of the stimulator 806.

Upon receiving the stimulation signal produced by the stimulator 806, the electrodes of the catheter 702 that are connected as stimulation electrodes (e.g., 724, 725) can apply electrical energy to a patient’s nerves through the biological lumen wall based on the received signal. Such stimulus can have any of a variety of known waveforms, such as a sinusoid, a square wave form or a triangular wave form, but is not limited thereto. In various examples, the stimulation can be applied for durations between approximately 0.05 milliseconds (msec) and approximately 8 msec.

The stimulation of nerves can be performed to evoke an elicited potential, which can cause such a potential to propagate in every direction along the nerve fibers. More generally, the STIM subsystem 805 can be used to deliver electrical stimulation via a selected pair of electrodes (e.g., 724, 725) in order to evoke a neural response, and the SENS subsystem 804 can be used to sense the evoked neural response.

In some embodiments, the ECU 802 can digitally sample the signal sensed using a pair of electrodes (e.g., 726, 727) to receive the electrical signal from the catheter 702. In alternate embodiments, the signal can be recorded as an analog signal. When receiving an electrical signal from the electrodes (e.g., 726, 727) on the catheter 702, the ECU 802 can perform filtering and/or other processing steps on the signal. Generally, such steps can be performed to discriminate the signal of interest sensed by the catheter (e.g., 702) from any background noise within the patient’s vasculature such that the resulting output is predominantly the signal from nerve cell activation. In some instances, the ECU 802 can modulate the electrical impedance of the signal receiving portion in order to accommodate the electrical properties and spatial separation of the electrodes mounted on the catheter in a manner to achieve the highest fidelity, selectively and resolution for the signal received. For example, electrode size, separation, and conductivity properties can impact the field strength at the electrode/tissue interface.

Additionally or alternatively, the ECU 802 can comprise a headstage and/or an amplifier 812 to perform any of offsetting, filtering, and/or amplifying the signal received from the catheter 702. In some examples, a headstage applies a DC offset to the signal and performs a filtering step. In some such systems, the filtering can comprise applying notch and/or band-pass filter(s) 814 to suppress particular undesired signals having a particular frequency content or to let pass desired signals having a particular frequency content. An amplifier 812 can be used to amplify the entire signal uniformly or can be used to amplify certain portions of the signal more than others. For example, in some configurations, the amplifier 812 can be configured to provide an adjustable capacitance of the recording electrode, changing the frequency dependence of signal pick-up and amplification. In some embodiments, properties of the amplifier 812, such as capacitance, can be adjusted to change amplification properties, such as the resonant frequency, of the amplifier.

In the illustrated embodiment of FIG. 8, the ECU 802 includes an amplifier 812 including a non-inverting (+) input terminal, an inverting (-) input terminal, a power supply input terminal, and a ground or reference terminal. As can be appreciated from FIG. 8, the non-inverting (+) input terminal can be coupled to the electrode 726, the inverting (-) input terminal can be electrically coupled to the electrode 727, the power supply input terminal is electrically coupled to a voltage source (e.g., a reference voltage generator), and the ground or reference terminal is electrically coupled to a ground reference electrode, which can be located on the catheter 702, can be located on a distal end of an introducer sheath, or can be located on the skin of the patient, but is not limited thereto.

In some embodiments, the ECU 802 can include a switching network configured to interchange which of electrodes (e.g., 724, 725, 726, 727) of a catheter (e.g., 702) are coupled to which portions of the ECU 802. In some such embodiments, a user can manually switch which inputs receive connections to which electrodes (e.g., 724, 725, 726, 727) of the catheter 702. Such configurability allows for a system operator to adjust the direction of propagation of the elicited potential as desired. For example, the switching network, or more generally switches, can be used to connect the electrodes 724 and 725 to the stimulator 806 during a period of time during which stimulation pulses are to be emitted by the catheter 702, and the switches can be used to connect the electrodes 726 and 726 to the amplifier 812 to sense the elicited response to the stimulation pulses. Additionally, or alternatively, a controller (e.g., 822) can autonomously control such a switching network.

The amplifier 812 can include any appropriate amplifier for amplifying desired signals or attenuating undesired signals. In some examples, the amplifier 812 has a high common-mode rejection ratio (CMRR) for eliminating or substantially attenuating undesired signals present at each of the sensing electrodes (e.g., 726, and 727). In some embodiments, the amplifier 812 can be adjusted, for example, via an adjustable capacitance or via other attributes of the amplifier.

In the example system 800 of FIG. 8, the ECU 802 further includes a filter 814 for enhancing the desired signal in the signal received via a pair of the electrodes (e.g., 726, 727). The filter 814 can include a band-pass filter, a notch filter, or any other appropriate filter to isolate desired signals from noise artifacts within the received signals. In some embodiments, various properties of the filter 814 can be adjusted to manipulate its filtering characteristics. For example, the filter may include an adjustable capacitance or other parameter to adjust its frequency response.

At least one of amplification and filtering of a sensed signal (e.g., received at the electrodes 726 and 727) can allow for extraction of the desired signal at 816. In some embodiments, extraction 816 comprises at least one additional processing step to isolate desired signals from the signal sensed using electrodes such as preparing the signal for output at 818. In some embodiments, the functionalities of any combination of amplifier 812, filter 814, and extraction 816 may be combined into a single entity. For instance, the amplifier 812 may act to filter undesired frequency content from the signal without requiring additional filtering at a separate filter.

In some embodiments, the ECU 802 can record emitted stimuli and/or received signals. Such data can be subsequently stored in permanent or temporary memory 820. The ECU 802 can comprise such memory 820 or can otherwise be in communication with external memory (not shown). Thus, the ECU 802 can be configured to emit stimulus pulses to electrodes (e.g., 724, 725) of the catheter (e.g., 702), record such pulses in a memory, receive signals from the catheter (e.g., 702), and also record such received signal data. The memory 820 in or associated with the ECU 802 can be internal or external to any part of the ECU 802 or the ECU 802 itself.

The ECU 802 or separate external processor can further perform calculations on the stored data to determine characteristics of signals either emitted or received via the catheter. For example, in various embodiments, the ECU 802 can determine any of the amplitude, duration, or timing of occurrence of the received or emitted signals. The ECU 802 can further determine the relationship between the received signal and the emitted stimulus signal, such as a temporal relationship therebetween. In some embodiments, the ECU 802 performs signal averaging on the signal data received from the catheter. Such averaging can act to reduce random temporal noise in the data while strengthening the data corresponding to any elicited potentials received by the catheter.

Averaging as such can result in a signal in which temporally random noise is generally averaged out and the signal present in each recorded data set, such as elicited potentials, will remain high. In some embodiments, each iteration of the process can include a synchronization step so that each acquired data set can be temporally registered to facilitate averaging the data. That is, events that occur consistently at the same time during each iteration may be detected, while temporally random artifacts (e.g., noise) can be reduced. In general, the signal to noise ratio (SNR) resulting in such averaging will improve by the square root of the number of samples averaged in order to create the averaged data set.

The ECU 802 can further present information regarding any or all of the applied stimulus, the signal, and the results of any calculations to a user of the system, e.g., via output 818. For example, the ECU 802 can generate a graphical display providing one or more graphs of signal strength vs. time representing the stimulus and/or the received signal.

In some embodiments, the ECU 802 can include a controller 822 in communication with one or both of stimulator 806 and SENS subsystem 804. The controller 822 can be configured to cause stimulator 806 to apply a stimulation signal to a catheter, e.g., the catheter 702. Additionally or alternatively, the controller 822 can be configured to analyze signals received and/or output by the SENS subsystem 804. In some embodiments, the controller 822 can act to control the timing of applying the stimulation signal from stimulator 806 and the timing of receiving signals by the SENS subsystem 804. The controller 822 can be implemented, e.g., using one or more processors, field programmable gate arrays (FPGAs), state machines, and/or application specific integrated circuits (ASICs), but is not limited thereto.

Example electrical control units have been described. In various embodiments, the ECU 802 can emit stimulus pulses to the catheter 702, receive signals from the catheter 702, perform calculations on the emitted and/or received signals, and present the signals and/or results of such calculations to a user. In some embodiments, the ECU 802 can comprise separate modules for emitting, receiving, calculating, and providing results of calculations. Additionally or alternatively, the functionality of controller 822 can be integrated into the ECU 802 as shown, or can be separate from and in communication with the ECU 802.

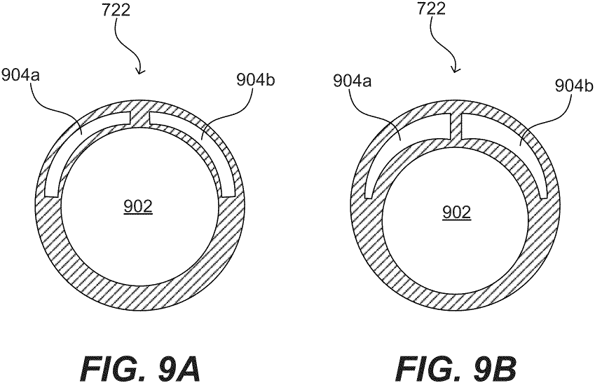

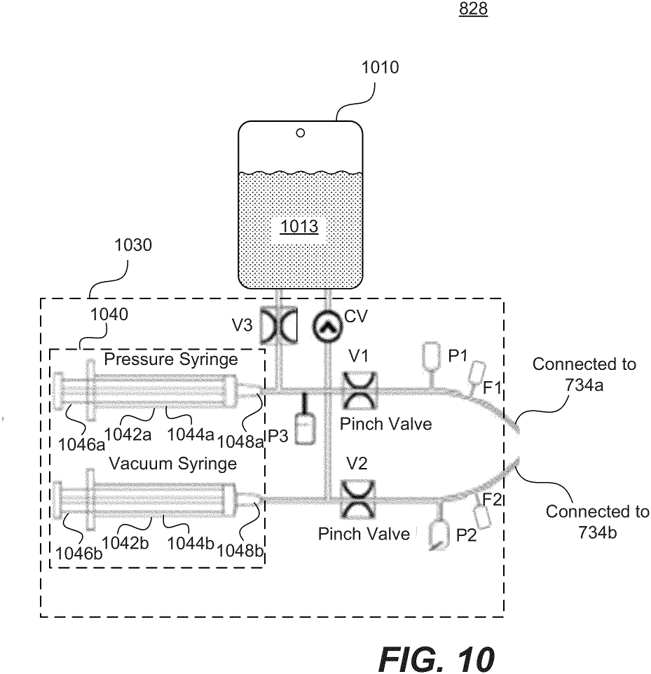

The controller 822 can also control a fluid supply subsystem 828, which can include a cartridge and a reservoir, which are described below with reference to FIG. 10, but can include alternative types of fluid pumps, and/or the like. The fluid supply subsystem 828 is fluidically coupled to one or more fluid lumens (e.g., 904a, 904b, in FIGS. 9A, 9B) within the catheter shaft 722 which in turn are fluidically coupled to the balloon 713. The fluid supply subsystem 828 can be configured to circulate a cooling liquid through the catheter 702 to the transducer 711 in the balloon 713.

### EXAMPLE CROSS-SECTION OF PORTION OF SHAFT OF CATHETER

Example cross-sections of a portion of the shaft 722 is shown in FIGS. 9A and 9B. Referring to FIG. 9A, the cross-section is shown as including a main lumen 902 having a circular cross-section, and smaller lumens 904a, 904b. In order to enable the fluid to be circulated through the balloon 713, the lumen 904a is fluidically coupled to the fluidic inlet port 734a (shown in FIG. 7) to enable fluid (e.g., expelled from a pressure syringe) to be provided to and at least partially fill the balloon 713, and the lumen 904b is fluidically coupled to the fluidic outlet port 734b (shown in FIG. 7) to enable fluid to be drawn from the balloon 713 (e.g., using a vacuum syringe). FIG. 9B shows alternative cross-sections for the lumens 902, 904b, and 904c. The main lumen 902 can function as a guide wire lumen, or the main lumen can be subdivided into additional lumen, one of which can be a guide wire lumen, and another of which can be a cable lumen that is used to hold electrical cabling that is electrically coupled to a transducer (e.g., 711). Other variations are also possible and within the scope of the embodiments described herein.

### EXAMPLE FLUID SUPPLY SUBSYSTEM

Example details of the fluid supply subsystem 828, which were introduced above in the discussion of FIG. 8, will now be described with reference to FIG. 10. Referring to FIG. 10, the fluid supply subsystem 828 is shown as including a cartridge 1030 and a reservoir 1010. The reservoir 1010 is shown as being implemented as a fluid bag, which can be the same or similar to an intravenous (IV) bag in that it can hang from a hook, or the like. The reservoir 1010 and the cartridge 1030 can be disposable and replaceable items.

The reservoir 1010 is fluidically coupled to the cartridge 1030 via a pair of fluidic paths, one of which is used as a fluid outlet path (that provides fluid from the reservoir to the cartridge), and the other one of which is used as a fluid inlet path (the returns fluid from the cartridge to the reservoir). The cartridge 1030 is shown as including a syringe pump 1040, which includes a pressure syringe 1042a and a vacuum syringe 1042b. The pressure syringe 1042a includes a barrel 1044a, a plunger 1046a, and a hub 1048a. Similarly, the vacuum syringe 1042b includes a barrel 1044b, a plunger 1046b, and a hub 1048b. The hub 1048a, 1048b of each of the syringes 1042a, 1042b is coupled to a respective fluid tube or hose. The cartridge 1030 is also shown as including pinch valves V1, V2 and V3, pressure sensors P1, P2, and P3, and a check valve CV. While not specifically shown in FIG. 10, the syringe pump 1040 can include one or more gears and step-motors, and/or the like, which are controlled by the controller 822 (in FIG. 8) to selectively maneuver the plungers 1046 of the pressure syringe 1042a and the vacuum syringe 1042b. Alternatively, the gear(s) and/or step-motor(s) can be used to control the syringe pump 1040.

In order to at least partially fill the barrel of the pressure syringe 1042a with a portion of the fluid that is stored in the reservoir 1010, the pinch valves V1 and V2 are closed, the pinch valve V3 is opened, and the plunger 1046a of the pressure syringe 1042a is pulled upon to draw fluid 1013 into the barrel 1044a of the of the pressure syringe 1042a. The pinch valve V3 is then closed and the pinch valves V1 and V2 are opened, and then the plunger 1046a of the pressure syringe 1042a is pushed upon to expel fluid from the barrel 1044a of the pressure syringe 1042a through the fluid tube attached to the hub 1048a of the pressure syringe 1042a. The fluid expelled from the pressure syringe 1042a enters a fluid lumen (e.g., 904a in the catheter shaft 722), via the fluidic inlet port 734a of the catheter 702, and then enters and at least partially fills the balloon 713. Simultaneously, the plunger 1046b of the vacuum syringe 1042b can be pulled upon to pull or draw fluid from the balloon 713 into a fluid lumen (e.g., 904b in the catheter shaft 722), through the fluidic outlet port 734b of the catheter 702, and then through fluid tube attached to the hub 1048b of the vacuum syringe 1042b and into the barrel 1044b of the vacuum syringe 1042b. In this manner, the fluid can be circulated through the balloon 713. The balloon 713 can be inflated by supplying more fluid to the balloon than is removed from the balloon. One or more of the pressure sensors P1, P2, and P3 can be used to monitor the pressure in the balloon 713 to achieve a target balloon pressure, e.g., of 70 pounds per square inch (psi), but not limited thereto. Once the balloon 713 is inflated to a target pressure, e.g., 70 psi, and/or size, the fluid can be circulated through the balloon 713 without increasing or decreasing the amount of fluid within the balloon by causing the same amount of fluid that is removed from the balloon 713 to be the same as the amount of fluid that is provided to the balloon 713. Also, once the target balloon pressure is reached, the ultrasound transducer 711 can be excited to emit ultrasound energy to treat tissue that surrounds the portion of the biological lumen (e.g., a portion of a renal artery) in which the balloon 713 and the transducer 711 are inserted. When the ultrasound transducer 711 is emitting ultrasound energy it can also be said that the ultrasound transducer 711 is performing sonication, or that sonication is occurring. During the sonication, cooling fluid should be circulated through the balloon 713 by continuing to push on the plunger 1046a of the pressure syringe 1042a and continuing to pull on the plunger 1046b of the vacuum syringe 1042b.

After the sonication is completed, and the balloon 713 is to be deflated so that the catheter 702 can be removed from the biological lumen, the cooling fluid should be returned from the barrel 1044b of the vacuum syringe 1042b to the reservoir 1010. In order to return the cooling fluid from the barrel 1044b of the vacuum syringe 1042b to the reservoir 1010, the pinch valves V1, V2, and V3 are all closed, and the plunger of the vacuum syringe 1042b is pushed on to expel the cooling fluid out of the barrel of the vacuum syringe 1042b, past the check valve CV, and into the reservoir 1010.

The pressure sensors P1, P2, and P3 can be used to monitor the fluidic pressure at various points along the various fluidic paths within the cartridge 1030, which pressure measurements can be provided to the controller 822 as feedback that is used for controlling the syringe pump 1040 and/or for other purposes, such as, but not limited to, determining the fluidic pressure within the balloon 713. Additionally, flow rate sensors F1 and F2 can be used, respectively, to monitor the flow rate of the fluid that is being injected (aka pushed, provided, or supplied) into the balloon 713, and to monitor the flow rate of the fluid that is being drawn (aka pulled or removed) from the balloon 713. The pressure measurements obtained from the pressure sensors P1, P2, and P3 can be provided to the controller 822 so that the controller 822 can monitor the balloon pressure. Additionally, flow rate measurements obtained from the flow rate sensors F1 and F2 can be provided to the controller 822 so that the controller 822 can monitor the flow rate of fluid being pushed into and pulled from the balloon 713. It would also be possible for one or more pressure sensors and/or flow rate sensors to be located at additional or alternative locations along the fluidic paths that provide fluid to and from the balloon 713.

### EXAMPLE TRANSDUCER

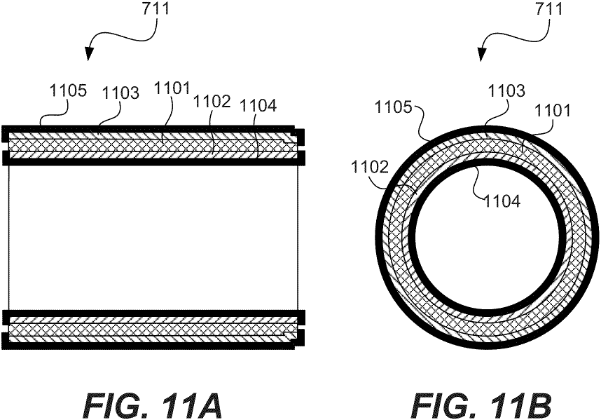

FIGS. 11A and 11B illustrate, respectively, a longitudinal cross-sectional view and a radial cross-sectional view of an example transducer 711 that can be physically coupled to one the tubes (e.g., the third tube 733) of the catheter 702 introduced above in the discussion of FIGS. 7A and 7B, in accordance with certain embodiments of the present technology. The transducer 711, which in the embodiment show in FIGS. 11A and 11B is an ultrasound transducer, includes a piezoelectric transducer body 1101 that comprises a hollow tube of piezoelectric material having an inner surface and an outer surface, with an inner electrode 1102 disposed on the inner surface of the hollow tube of piezoelectric material, and an outer electrode 1103 disposed on the outer surface of the hollow tube of piezoelectric material. The hollow tube of piezoelectric material, or more generally the piezoelectric transducer body 1101, is cylindrically shaped and has a circular radial cross-section. However, in alternative embodiments the transducer body 1101 can have other shapes besides being cylindrical with a circular radial cross-section. The inner electrode 1102 is covered by an electrical insulator 1104, and the outer electrode 1103 is covered by an electrical insulator 1105. It is also possible that only one of the electrodes 1102, 1103 is covered by an electrical insulator, or that neither of the electrodes 1102, 1103 is covered by an electrical insulator. Other variations are also possible and within the scope of the embodiments described herein.

### SYSTEM

An aspect of the invention relates to a system 800 for performing a denervation procedure and determining an efficacy thereof. The system 800 comprises a catheter 302, 702 including an element 311, 711 or first and second elements 311, 711 configured to selectively deliver ablation therapy and one or more sensing electrodes 726, 727 configured to selectively sense neural activity. The system 800 also comprises an excitation source 826 configured to selectively energize the element 311, 711 or the first and second elements 311, 711 of the catheter 302, 702 to thereby cause the element 311, 711 or the first and second elements 311, 711 to deliver the ablation therapy. The system 800 further comprises a sensing subsystem 804 electrically coupled to the at least one sensing electrode 726, 727. The system 800 additionally comprises a controller 822 communicatively coupled to the catheter 302, 702, the excitation source 826 and the sensing subsystem 804. The controller 822 is configured to cause the excitation source 826 to energize the element 311, 711 or the first element 311, 711 to thereby delivery first ablation therapy, while the element 311, 711 or the first element 311, 711 is located at a first longitudinal location along a biological lumen. The controller 822 is also configured to cause the excitation source 826 to energize the element 311, 711 or the second element 311, 711 to thereby delivery second ablation therapy, while the element 311, 711 or the second element 311, 711 is located at a second longitudinal location along the biological lumen 104 that is longitudinally spaced apart from the first longitudinal location. The controller 822 is further configured to cause the sensing subsystem 804 to sense neural activity from a third longitudinal location, using at least one of the one or more sensing electrodes 726, 727, to thereby quantify to what extent nerves surrounding the biological lumen 104 and adjacent to at least one of the first and the second longitudinal locations were affected by the delivery of the first and the second ablation therapy.

In an embodiment, the system 800 also comprises memory 820 configured to store instructions executable by the one or more processors of the controller 822 to cause the excitation source 826 to energize the element 311, 711 or the first and second elements 311, 711 and to cause the sensing subsystem 814 to sense neural activity.

In an embodiment, the neural activity that is sensed using the sensing subsystem 804 comprises native neural activity.

In an embodiment, the system 800 further comprises one or more stimulation electrodes 724, 725 located on the catheter 302, 702, and a stimulator 806 electrically coupled to the one or more stimulation electrodes 724, 725 and communicatively coupled to the controller 822. In this embodiment, the controller 822 is further configured to cause the stimulator 806 to produce one or more stimulation pulses that are delivered via at least one of the one or more stimulation electrodes 724, 725 to thereby evoke a neural response. The neural activity that is sensed by the sensing subsystem 804 from the third longitudinal location comprises an evoked neural response to the one or more stimulation pulses.

In an embodiment, the third longitudinal location, from which the neural activity is sensed, is longitudinally located between the first and the second longitudinal locations, from which the first and the second ablation therapy were respectively delivered.

In an embodiment, at least one of the one or more stimulation electrodes 724, 725) used to deliver the one or more stimulation pulses, is located at a longitudinal location that is proximal to both the first and the second longitudinal locations. In this embodiment, the third location, from which the evoked neural response is sensed using at least one of the one or more sensing electrodes 726, 727, is distal to both the first and the second longitudinal locations.

In an embodiment, at least one of the one or more stimulation electrodes 724, 725, used to deliver the one or more stimulation pulses, is located at a longitudinal location that is distal to both the first and the second longitudinal locations. In this embodiment, the third location, from which the evoked neural response is sensed using at least one of the one or more sensing electrodes 726, 727, is proximal to both the first and the second longitudinal locations.

In an embodiment, the element 311, 711 comprises a transducer 311, 711.

In an embodiment, the controller 822 is configured to cause to the excitation source 826 to energize the transducer 311, 711 to deliver the first ablation therapy from the first longitudinal location along the biological lumen 104 and energize the transducer 311, 711 to deliver the second ablation therapy from the second longitudinal location longitudinally spaced apart from the first longitudinal location along the biological lumen 104 after the transducer 311, 711 is moved from the first longitudinal location to the second longitudinal location.

In an embodiment, the catheter 302, 702 comprises first and second transducers 311, 711 longitudinally spaced apart from one another along a shaft 722 of the catheter 302, 702. In this embodiment, the controller 822 is configured to cause to the excitation source 826 to energize the first transducer 311, 711 to deliver the first ablation therapy from the first longitudinal location along the biological lumen 104 and energize the second transducer 311, 711 to deliver the second ablation therapy from the second longitudinal location longitudinally spaced apart from the first longitudinal location along the biological lumen 104.

In an embodiment, the system 800 further comprises one or more stimulation electrodes 724, 725 located on the catheter 302, 702, and a stimulator 806 electrically coupled to the one or more stimulation electrodes 724, 725 and communicatively coupled to the controller 822. In this embodiment, the controller 822 is further configured to cause the stimulator 806 to produce one or more stimulation pulses that are delivered via at least one of the one or more stimulation electrodes 724, 725 at a longitudinal location that is one of proximal or distal to both the first and second longitudinal locations to thereby evoke a neural response. The neural activity that is sensed by the sensing subsystem 804 from the third longitudinal location comprises an evoked neural response to the one or more stimulation pulses and the third longitudinal location is the other one of proximal or distal to both the first and second longitudinal locations.

In an embodiment, the element 311, 711 or the first and second element 311, 711 comprise(s) one or more electrodes.

In an embodiment, the controller 822 is configured to determine, based on the neural activity sensed from the third longitudinal location, whether or not further ablation therapy should be delivered from a location along the longitudinal length of the biological lumen 104.

In an embodiment, the controller 822 is configured to compare a measure of the neural activity that is sensed to a corresponding threshold. The controller 822 is, in this embodiment, also configured to determine that the further ablation therapy should be delivered when the measure of the neural activity that is sensed exceeds the corresponding threshold. The controller 822, in an embodiment, is also configured to determine that ablation therapy is complete when the measure of the neural activity that is sensed is less than the corresponding threshold.

In an embodiment, the controller 822 is configured to compare a measure of the neural activity that is sensed to multiple thresholds, e.g., at least a first and second threshold. In an embodiment, the controller 822 determines that the ablation is incomplete when the neural activity is less than the first threshold but more than the second threshold.

In an embodiment, a user interface 824 may be used to display a comparison between a measured neural activity and a threshold.

In an embodiment, a user interface 824 may be used to display a message to a user that the ablation was successful, aka complete, incomplete, and/or unsuccessful based on the determination of the controller 822.

In an embodiment, a user interface 824 may prompt the user to move to a different location after the controller 822 has determined that the ablation is successful.

In an embodiment, a user interface 824 may prompt the user to either move to a different location after the controller 822 has determined that the ablation is unsuccessful and/or the user interface 824 may display a message that the ablation is incomplete and/or the user interface 824 may prompt the user to keep ablating at the same locations.

In an embodiment, the controller 822 is configured to compare the number of complete ablations against a preprogrammed number of complete ablations to determine whether treatment is complete, and no further ablations need be performed. For example, the preprogrammed number of complete ablations may be 2 or 3 ablations per main renal artery, and the controller 822 may determine that treatment is complete once the controller determines that there have been 2 or 3 complete ablations per main renal artery.

In an embodiment, the controller 822 is configured to cause, in response to the controller 822 determining that further ablation therapy should be delivered, the excitation source 826 to energize the element 311, 711 or the first or second element 311, 711 to thereby delivery further ablation therapy, while the element 311, 711 or the first or second element 311, 711 is located at the location along a longitudinal length of the biological lumen 104.

In an embodiment, the element 311, 711 or the first or second element 311, 711 is configured to deliver one of the following types of ablation therapy: radio frequency (RF) energy, microwave energy, ultrasound energy, pulsed electric field energy, optical energy, laser light, cryotherapeutic therapy, or a chemical agent.

### EXAMPLE SYSTEMS AND METHODS

Example 1. A system for performing a denervation procedure and determining an efficacy thereof, comprising: a catheter including one or more elements configured to selectively deliver ablation therapy and one or more sensing electrodes configured to selectively sense neural activity; an excitation source configured to selectively energize at least one of the one or more elements of the catheter to thereby cause at least one of the one or more elements to deliver the ablation therapy; a sensing subsystem electrically coupled to at least one of the one or more sensing electrodes; and a controller communicatively coupled to the catheter, the excitation source and the sensing subsystem, wherein the controller is configured to: cause the excitation source to energize at least one of said elements to thereby delivery first ablation therapy, while the said element is located at a first longitudinal location along a biological lumen; cause the excitation source to energize at least one of said elements to thereby delivery second ablation therapy, while the said element is located at a second longitudinal location along the biological lumen that is longitudinally spaced apart from the first longitudinal location; and cause the sensing subsystem to sense neural activity from a third longitudinal location, using at least one of the one or more sensing electrodes, to determine the efficacy of at least one of the first or second ablation therapies.

Example 2. The system of example 1, wherein the third longitudinal location is longitudinally located between the first and the second longitudinal locations, from which the first and the second ablation therapy are respectively delivered.

Example 3. The system of any one of examples 1 or 2, wherein the neural activity that is sensed using the sensing subsystem comprises native neural activity.

Example 4. The system of example 1, further comprising: one or more stimulation electrodes located on the catheter; and a stimulator electrically coupled to the one or more stimulation electrodes and communicatively coupled to the controller; wherein the controller is further configured to cause the stimulator to produce one or more stimulation pulses that are delivered via at least one of the one or more stimulation electrodes to thereby evoke a neural response; and wherein the neural activity that is sensed by the sensing subsystem from the third longitudinal location comprises an evoked neural response to the one or more stimulation pulses.

Example 5. The system of example 4, wherein the third longitudinal location, from which the evoked neural response is sensed, is longitudinally located between the first and the second longitudinal locations, from which the first and the second ablation therapy were respectively delivered.

Example 6. The system of example 4, wherein: at least one of the one or more stimulation electrodes, used to deliver the one or more stimulation pulses, is located at a longitudinal location that is proximal to both the first and the second longitudinal locations; and the third location, from which the evoked neural response is sensed using at least one of the one or more sensing electrodes, is distal to both the first and the second longitudinal locations.

Example 7. The system of example 4, wherein: at least one of the one or more stimulation electrodes, used to deliver the one or more stimulation pulses, is located at a longitudinal location that is distal to both the first and the second longitudinal locations; and the third location, from which the evoked neural response is sensed using at least one of the one or more sensing electrodes, is proximal to both the first and the second longitudinal locations.

Example 8. The system of any one of examples 1 through 7, wherein the one or more elements comprise a transducer.

Example 9. The system of example 8, wherein the controller is configured to cause to the excitation source to energize the transducer to deliver the first ablation therapy from the first longitudinal location along the biological lumen and energize the transducer to deliver the second ablation therapy from the second longitudinal location longitudinally spaced apart from the first longitudinal location along the biological lumen after the transducer is moved from the first longitudinal location to the second longitudinal location.

Example 10. The system of any one of examples 1 through 7, wherein: the one or more elements comprise a first transducer and a second transducer longitudinally spaced apart from the first transducer; and the controller is configured to cause to the excitation source to energize the first transducer to deliver the first ablation therapy from the first longitudinal location along the biological lumen and energize the second transducer to deliver the second ablation therapy from the second longitudinal location longitudinally spaced apart from the first longitudinal location along the biological lumen.

Example 11. The system of example 1, further comprising: one or more stimulation electrodes located on the catheter; and a stimulator electrically coupled to the one or more stimulation electrodes and communicatively coupled to the controller; wherein the controller is further configured to cause the stimulator to produce one or more stimulation pulses that are delivered via at least one of the one or more stimulation electrodes at a longitudinal location that is one of proximal or distal to both the first and second longitudinal locations to thereby evoke a neural response; and wherein the neural activity that is sensed by the sensing subsystem from the third longitudinal location comprises an evoked neural response to the one or more stimulation pulses and the third longitudinal location is the other one of proximal or distal to both the first and second longitudinal locations.

Example 12. The system of any one of examples 1 through 11, wherein the controller is configured to determine, based on the neural activity sensed from the third longitudinal location, whether or not further ablation therapy should be delivered from a location along a longitudinal length of the biological lumen.

Example 13. The system of example 12, wherein the controller is configured to: compare a measure of the neural activity that is sensed to a corresponding threshold; and determine that the further ablation therapy should be delivered when the measure of the neural activity that is sensed exceeds the corresponding threshold.

Example 14. The system of example 13, wherein the controller is configured to cause, in response to the controller determining that further ablation therapy should be delivered, the excitation source to energize at least one of the one or more elements to thereby delivery further ablation therapy, while the at least one of the one or more elements is located at the location along a longitudinal length of the biological lumen.

Example 15. The system of any one of examples 1 through 7 or 11 through 14, wherein each of the one or more elements is configured to deliver one of the following types of ablation therapy: radio frequency (RF) energy; microwave energy; ultrasound energy; pulsed electric field energy; optical energy; laser light; cryotherapeutic therapy; or a chemical agent.

Example 16. A method for performing a denervation procedure and determining an efficacy thereof, comprising: delivering first ablation therapy from a first longitudinal location along a biological lumen; delivering second ablation therapy from a second longitudinal location longitudinally spaced apart from the first longitudinal location along the biological lumen; and sensing neural activity from a third longitudinal location that is longitudinally located between the first and the second longitudinal locations, from which the first and the second ablation therapy were respectively delivered, to determine the efficacy of at least one of the first or second ablation therapies.

Example 17. The method of example 16, wherein the method is performed using a catheter including an element configured to selectively deliver ablation therapy, and wherein the method further comprises: inserting a distal portion of the catheter into the biological lumen such that the element is located at the first longitudinal location along the biological lumen, wherein the delivering the first ablation therapy, using the element, is performed while the element is located at the first longitudinal location along the biological lumen; and maneuvering the catheter such that the element is located at the second longitudinal location longitudinally spaced apart from the first longitudinal location along the biological lumen, wherein the delivering the second ablation therapy, using the element, is performed while the element is located at the second longitudinal location along the biological lumen.

Example 18. The method of example 17, wherein the element comprises a transducer.

Example 19. The method of example 17, wherein the element comprises one or more electrodes.

Example 20. The method of example 16, wherein the method is performing using a catheter including first and second transducers longitudinally spaced apart from one another along a shaft of the catheter, and wherein: the delivering the first ablation therapy from the first longitudinal location along the biological lumen is performing using the first transducer; and the delivering the second ablation therapy from the second longitudinal location longitudinally spaced apart from the first longitudinal location along the biological lumen is performed using the second transducer.

Example 21. The method of any one of examples 16 through 20, further comprising: determining, based on the neural activity sensed from the third longitudinal location, whether or not further ablation therapy should be delivered from a location along a longitudinal length of the biological lumen.

Example 22. The method of example 21, wherein the determining, based on the neural activity sensed from the third longitudinal location, whether or not the further ablation therapy should be delivered, comprises: comparing a measure of the neural activity that is sensed to a corresponding threshold; and determining that the further ablation therapy should be delivered, when the measure of the neural activity that is sensed exceeds the corresponding threshold.

Example 23. The method of example 22, further comprising: delivering the further ablation therapy in response to the determining that the further ablation therapy should be delivered.

Example 24. The method of example 16, wherein: the sensing the neural activity from the third longitudinal location is performed using an electrode positioned at the third longitudinal location that is longitudinally located between the first and the second longitudinal locations; and the electrode, which is used for the sensing the neural activity from the third longitudinal location, is included on a distal portion of a catheter, which can be a same catheter as or a different catheter than is used for the delivering the first and the second ablation therapy.

Example 25. The method of example 24, wherein: the neural activity that is sensed from the third longitudinal location comprises native neural activity that is sensed using the electrode positioned at the third longitudinal location; the native neural activity is also sensed using a further electrode; and the further electrode is either positioned within the biological lumen or externally on skin.

Example 26. The method of example 25, wherein the further electrode is positioned within the biological lumen between the first and the second longitudinal locations.

Example 27. The method of example 16, wherein: the neural activity that is sensed from the third longitudinal location comprises an evoked neural response to stimulation that is delivered from an electrode positioned within the biological lumen.

Example 28. The method of example 27, wherein: the evoked neural response is also sensed using a further electrode; and the further electrode is either positioned within the biological lumen or externally on skin.

Example 29. The method of any one of examples 16 through 28, wherein the biological lumen comprises a renal artery.

Example 30. The method of any one of examples 16, 17 or 21 through 29, wherein each of the first and the second ablation therapy is performing by delivering one of the following, using a catheter including a shaft inserted into the biological lumen: radio frequency (RF) energy; microwave energy; ultrasound energy; pulsed electric field energy; optical energy; laser light; cryotherapeutic therapy; or a chemical agent.

Example 31. A method for performing a denervation procedure and determining an efficacy thereof, comprising: delivering first ablation therapy from a first longitudinal location along a biological lumen; delivering second ablation therapy from a second longitudinal location longitudinally spaced apart from the first longitudinal location along the biological lumen; delivering stimulation energy using an electrode that is located at a third longitudinal location along the biological lumen, wherein the third longitudinal location is one of proximal or distal to both the first and the second longitudinal locations; sensing an evoked neural response to the stimulation energy using a further electrode located at a fourth longitudinal location along the biological lumen, wherein the fourth longitudinal location is the other one of proximal or distal to both the first and the second longitudinal locations; and determining, based on the evoked neural response that is sensed, whether or not further ablation therapy should be delivered from a location along a longitudinal length of the biological lumen.

Example 32. The method of example 31, wherein: the third longitudinal location is proximal to both the first and the second longitudinal locations; and the fourth longitudinal location is distal to both the first and the second longitudinal locations.

Example 33. The method of example 31, wherein: the third longitudinal location is distal to both the first and the second longitudinal locations; and the fourth longitudinal location is proximal to both the first and the second longitudinal locations.

Example 34. The method of any one of examples 31 through 33, wherein: the delivering the first ablation therapy from the first longitudinal location is performing using a transducer of a distal portion of a catheter that is inserted into the biological lumen such that the transducer is positioned at the first longitudinal location; and the delivering the second ablation therapy from the second longitudinal location is performing using the transducer of the distal portion of the catheter that is inserted into the biological lumen, after the transducer is moved from being positioned at the first longitudinal location to being positioned at the second longitudinal location.

Example 35. The method of any one of examples 31 through 33, wherein: the delivering the first ablation therapy from the first longitudinal location is performing by emitting first radio frequency (RF) energy using one or more electrodes of a distal portion of a catheter that is inserted into the biological lumen such that the one or more electrodes are positioned at the first longitudinal location; and the delivering the second ablation therapy from the second longitudinal location is performing by emitting second RF energy using the one or more electrodes of the distal portion of the catheter that is inserted into the biological lumen, after the one or more electrodes are moved from being positioned at the first longitudinal location to being positioned at the second longitudinal location.

Example 36. The method of any one of examples 31 through 35, wherein the determining, based on the evoked neural response that is sensed, whether or not the further ablation therapy should be delivered, comprises: comparing a measure of the evoked neural response that is sensed to a corresponding threshold; and determining that the further ablation therapy should be delivered, when the measure of the evoked neural response that is sensed exceeds the corresponding threshold; and further comprising delivering the further ablation therapy in response to the determining that the further ablation therapy should be delivered.

Example 37. The method of any one of examples claims 31 through 33 or 36, wherein each of the first and the second ablation therapy is performing by delivering one of the following, using a catheter including a shaft inserted into the biological lumen: radio frequency (RF) energy; microwave energy; ultrasound energy; pulsed electric field energy; optical energy; laser light; cryotherapeutic therapy; or a chemical agent.

Although several embodiments and examples are disclosed herein, the present application extends beyond the specifically disclosed embodiments to other alternative embodiments and/or uses of the inventions and modifications and equivalents thereof. It is also contemplated that various combinations or subcombinations of the specific features and aspects of the embodiments may be made and still fall within the scope of the inventions. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above, but should be determined only by a fair reading of the claims that follow.

While the inventions are susceptible to various modifications, and alternative forms, specific examples thereof have been shown in the drawings and are herein described in detail. It should be understood, however, that the inventions are not to be limited to the particular forms or methods disclosed, but, to the contrary, the inventions are to cover all modifications, equivalents, and alternatives falling within the spirit and scope of the various embodiments described and the appended claims. Any methods disclosed herein need not be performed in the order recited.

What is claimed is:

1. A system for performing a denervation procedure and determining an efficacy thereof, comprising:
a catheter including one or more elements configured to selectively deliver ablation therapy and one or more sensing electrodes configured to selectively sense neural activity;
an excitation source configured to selectively energize at least one of the one or more elements of the catheter to thereby cause at least one of the one or more elements to deliver the ablation therapy;
a sensing subsystem electrically coupled to at least one of the one or more sensing electrodes; and
a controller communicatively coupled to the catheter, the excitation source and the sensing subsystem, wherein the controller is configured to:
cause the excitation source to energize at least one of said one or more elements to thereby deliver first ablation therapy at a first longitudinal location along a biological lumen;
cause the excitation source to energize at least one of said one or more elements to thereby deliver second ablation therapy at a second longitudinal location along the biological lumen that is longitudinally spaced apart from the first longitudinal location; and
cause the sensing subsystem to sense neural activity from a third longitudinal location using at least one of the one or more sensing electrodes to determine the efficacy of at least one of the first or second ablation therapies.

2. The system of claim 1, wherein the third longitudinal location is longitudinally located between the first and the second longitudinal locations, from which the first and the second ablation therapy are respectively delivered.

3. The system of claim 1, wherein the controller is configured to quantify to what extent nerves surrounding the biological lumen and adjacent to at least one of the first and the second longitudinal locations were affected by the delivery of the first and the second ablation therapy based on the neural activity sensed from the third longitudinal location.

4. The system of claim 1, further comprising:
one or more stimulation electrodes located on the catheter; and
a stimulator electrically coupled to the one or more stimulation electrodes and communicatively coupled to the controller;
wherein the controller is further configured to cause the stimulator to produce one or more stimulation pulses that are delivered via at least one of the one or more stimulation electrodes to thereby evoke a neural response; and
wherein the neural activity that is sensed by the sensing subsystem from the third longitudinal location comprises an evoked neural response to the one or more stimulation pulses.

5. The system of claim 4, wherein the third longitudinal location, from which the evoked neural response is sensed, is longitudinally located between the first and the second longitudinal locations, from which the first and the second ablation therapy were respectively delivered.

6. The system of claim 4, wherein:
at least one of the one or more stimulation electrodes, used to deliver the one or more stimulation pulses, is located at a longitudinal location that is proximal to both the first and the second longitudinal locations; and
the third location, from which the evoked neural response is sensed using at least one of the one or more sensing electrodes, is distal to both the first and the second longitudinal locations.

7. The system of claim 4, wherein:
at least one of the one or more stimulation electrodes, used to deliver the one or more stimulation pulses, is located at a longitudinal location that is distal to both the first and the second longitudinal locations; and
the third location, from which the evoked neural response is sensed using at least one of the one or more sensing electrodes, is proximal to both the first and the second longitudinal locations.

8. The system of claim 1, wherein the one or more elements comprise a transducer.

9. The system of claim 8, wherein the controller is configured to cause to the excitation source to energize the transducer to deliver the first ablation therapy from the first longitudinal location along the biological lumen and energize the transducer to deliver the second ablation therapy from the second longitudinal location longitudinally spaced apart from the first longitudinal location along the biological lumen after the transducer is moved from the first longitudinal location to the second longitudinal location.

10. The system of claim 1, wherein:
the one or more elements comprise a first transducer and a second transducer longitudinally spaced apart from the first transducer; and
the controller is configured to cause to the excitation source to energize the first transducer to deliver the first ablation therapy from the first longitudinal location along the biological lumen and energize the second transducer to deliver the second ablation therapy from the second longitudinal location longitudinally spaced apart from the first longitudinal location along the biological lumen.

11. The system of claim 1, further comprising:
one or more stimulation electrodes located on the catheter; and
a stimulator electrically coupled to the one or more stimulation electrodes and communicatively coupled to the controller;
wherein the controller is further configured to cause the stimulator to produce one or more stimulation pulses that are delivered via at least one of the one or more stimulation electrodes at a longitudinal location that is one of proximal or distal to both the first and second longitudinal locations to thereby evoke a neural response; and
wherein the neural activity that is sensed by the sensing subsystem from the third longitudinal location comprises an evoked neural response to the one or more stimulation pulses and the third longitudinal location is the other one of proximal or distal to both the first and second longitudinal locations.

12. The system of claim 1, wherein the controller is configured to determine, based on the neural activity sensed from the third longitudinal location, whether or not further ablation therapy should be delivered.

13. The system of claim 12, wherein the controller is configured to:
compare a measure of the neural activity that is sensed to a corresponding threshold; and
determine that the further ablation therapy should be delivered when the measure of the neural activity that is sensed exceeds the corresponding threshold.

14. The system of claim 13, wherein the controller is configured to cause, in response to the controller determining that further ablation therapy should be delivered, the excitation source to energize at least one of the one or more elements to thereby delivery further ablation therapy, while the at least one of the one or more elements is located at a location along a longitudinal length of the biological lumen.

15. The system of claim 1, wherein each of the one or more elements is configured to deliver one of the following types of ablation therapy:
radio frequency (RF) energy;
microwave energy;
ultrasound energy;
pulsed electric field energy;
optical energy;
laser light;
cryotherapeutic therapy; or
a chemical agent.

16. A method for performing a denervation procedure and determining an efficacy thereof, comprising:
delivering first ablation therapy from a first longitudinal location along a biological lumen;
delivering second ablation therapy from a second longitudinal location longitudinally spaced apart from the first longitudinal location along the biological lumen; and
sensing neural activity from a third longitudinal location that is longitudinally located between the first and the second longitudinal locations, from which the first and the second ablation therapy were respectively delivered to determine the efficacy of at least one of the first or second ablation therapies.

17. The method of claim 16, wherein the method is performed using a catheter including an element configured to selectively deliver ablation therapy, and wherein the method further comprises:
inserting a distal portion of the catheter into the biological lumen such that the element is located at the first longitudinal location along the biological lumen, wherein the delivering the first ablation therapy, using the element, is performed while the element is located at the first longitudinal location along the biological lumen; and
maneuvering the catheter such that the element is located at the second longitudinal location longitudinally spaced apart from the first longitudinal location along the biological lumen, wherein the delivering the second ablation therapy, using the element, is performed while the element is located at the second longitudinal location along the biological lumen.

18. The method of claim 17, wherein the element comprises a transducer.

19. The method of claim 17, wherein the element comprises one or more electrodes.

20. The method of claim 16, wherein the method is performing using a catheter including first and second transducers longitudinally spaced apart from one another along a shaft of the catheter, and wherein:
the delivering the first ablation therapy from the first longitudinal location along the biological lumen is performing using the first transducer; and
the delivering the second ablation therapy from the second longitudinal location longitudinally spaced apart from the first longitudinal location along the biological lumen is performed using the second transducer.

21. The method of claim 16, further comprising:
determining, based on the neural activity sensed from the third longitudinal location, whether or not further ablation therapy should be delivered.

22. The method of claim 21, wherein the determining, based on the neural activity sensed from the third longitudinal location, whether or not the further ablation therapy should be delivered, comprises:
comparing a measure of the neural activity that is sensed to a corresponding threshold; and
determining that the further ablation therapy should be delivered, when the measure of the neural activity that is sensed exceeds the corresponding threshold.

23. The method of claim 22, further comprising:
delivering the further ablation therapy in response to the determining that the further ablation therapy should be delivered.

24. The method of claim 16, wherein:
the sensing the neural activity from the third longitudinal location is performed using an electrode positioned at the third longitudinal location that is longitudinally located between the first and the second longitudinal locations; and
the electrode, which is used for the sensing the neural activity from the third longitudinal location, is included on a distal portion of a catheter, which can be a same catheter as or a different catheter than is used for the delivering the first and the second ablation therapy.

25. The method of claim 24, wherein:
the neural activity that is sensed from the third longitudinal location comprises native neural activity that is sensed using the electrode positioned at the third longitudinal location;
the native neural activity is also sensed using a further electrode; and
the further electrode is either positioned within the biological lumen or externally on skin.

26. The method of claim 25, wherein the further electrode is positioned within the biological lumen between the first and the second longitudinal locations.

27. The method of claim 16, wherein:
the neural activity that is sensed from the third longitudinal location comprises an evoked neural response to stimulation that is delivered from an electrode positioned within the biological lumen.

28. The method of claim 27, wherein:
the evoked neural response is also sensed using a further electrode; and
the further electrode is either positioned within the biological lumen or externally on skin.

29. The method of claim 16, wherein the biological lumen comprises a renal artery.

30. The method of claim 16, wherein each of the first and the second ablation therapy is performing by delivering one of the following, using a catheter including a shaft inserted into the biological lumen:
radio frequency (RF) energy;
microwave energy;
ultrasound energy;
pulsed electric field energy;
optical energy;
laser light;
cryotherapeutic therapy; or
a chemical agent.

31. A method for performing a denervation procedure and determining an efficacy thereof, comprising:
delivering first ablation therapy from a first longitudinal location along a biological lumen;
delivering second ablation therapy from a second longitudinal location longitudinally spaced apart from the first longitudinal location along the biological lumen;
delivering stimulation energy using an electrode that is located at a third longitudinal location along the biological lumen, wherein the third longitudinal location is one of proximal or distal to both the first and the second longitudinal locations;
sensing an evoked neural response to the stimulation energy using a further electrode located at a fourth longitudinal location along the biological lumen, wherein the fourth longitudinal location is the other one of proximal or distal to both the first and the second longitudinal locations; and
determining, based on the evoked neural response that is sensed, whether or not further ablation therapy should be delivered.

32. The method of claim 31, wherein:
the third longitudinal location is proximal to both the first and the second longitudinal locations; and
the fourth longitudinal location is distal to both the first and the second longitudinal locations.

33. The method of claim 31, wherein:
the third longitudinal location is distal to both the first and the second longitudinal locations; and
the fourth longitudinal location is proximal to both the first and the second longitudinal locations.

34. The method of claim 31, wherein:
the delivering the first ablation therapy from the first longitudinal location is performing using a transducer of a distal portion of a catheter that is inserted into the biological lumen such that the transducer is positioned at the first longitudinal location; and
the delivering the second ablation therapy from the second longitudinal location is performing using the transducer of the distal portion of the catheter that is inserted into the biological lumen, after the transducer is moved from being positioned at the first longitudinal location to being positioned at the second longitudinal location.

35. The method of claim 31, wherein:
the delivering the first ablation therapy from the first longitudinal location is performing by emitting first radio frequency (RF) energy using one or more electrodes of a distal portion of a catheter that is inserted into the biological lumen such that the one or more electrodes are positioned at the first longitudinal location; and
the delivering the second ablation therapy from the second longitudinal location is performing by emitting second RF energy using the one or more electrodes of the distal portion of the catheter that is inserted into the biological lumen, after the one or more electrodes are moved from being positioned at the first longitudinal location to being positioned at the second longitudinal location.

36. The method of claim 31, wherein the determining, based on the evoked neural response that is sensed, whether or not the further ablation therapy should be delivered, comprises:
comparing a measure of the evoked neural response that is sensed to a corresponding threshold; and
determining that the further ablation therapy should be delivered, when the measure of the evoked neural response that is sensed exceeds the corresponding threshold; and further comprising delivering the further ablation therapy in response to the determining that the further ablation therapy should be delivered.

37. The method of claim 31, wherein each of the first and the second ablation therapy is performing by delivering one of the following, using a catheter including a shaft inserted into the biological lumen:
radio frequency (RF) energy;
microwave energy;
ultrasound energy;
pulsed electric field energy;
optical energy;
laser light;
cryotherapeutic therapy; or
a chemical agent.

38. The method of claim 16, further comprising quantifying to what extent nerves surrounding the biological lumen and adjacent to at least one of the first and the second longitudinal locations were affected by the delivery of the first and the second ablation therapy based on the neural activity sensed from the third longitudinal location.

39. The method of claim 16, further comprising displaying on a user interface a message to a user that the at least one of the first or second ablation therapies were successful, incomplete, and/or unsuccessful based on the neural activity sensed from the third longitudinal location.

40. The system of claim 1, wherein the neural activity that is sensed using the sensing subsystem comprises native neural activity.

41. The system of claim 3, further comprising a user interface configured to display a message to a user that the ablation was successful, incomplete, and/or unsuccessful based on the determination of the controller.
